(19) United States
(12) Patent Application Publication
(10) Pub. No.: US 2006/0246095 A1
(43) Pub. Date: Nov. 2, 2006

(54) Multiepitope polypeptides for cancer immunotherapy

(75) Inventors: Tamar Peretz, Jerusalem (IL); Michal Lotem, Macabim (IL); Shoshana Frankenburg, Jerusalem (IL); Jacob Pitcovski, Korazim (IL); Adva Levi, Kiryat Shmona (IL); Hanah Margalit, Jerusalem (IL); Yael Altuvia, Jerusalem (IL)

Correspondence Address: FLEIT KAIN GIBBONS GUTMAN BONGINI & BIANCO, 21355 EAST DIXIE HIGHWAY, SUITE 115, MIAMI, FL, 33180, US

(73) Assignees: HADASIT MEDICAL RESEARCH SERVICES & DEVELOPMENT LTD., JERUSALEM (IL); YISSUM RESEARCH DEVELOPMENT COMPANY OF THE HEBREW UNIVERSITY OF JERUSALEM, JERUSALEM (IL); GAVISH-GALILEE-BIO APPLICATIONS LTD., KIBBUTZ GALIL-YAM (IL)

(21) Appl. No.: 11/388,794

(22) Filed: Mar. 24, 2006

Related U.S. Application Data
(63) Continuation-in-part of application No. PCT/IL04/00894, filed on Sep. 26, 2004.

(30) Foreign Application Priority Data
Sep. 25, 2003 (IL) 158140

(51) Int. Cl. A61K 39/00; G01N 33/574; C07H 21/04; C12P 21/06; C07K 14/82

(52) U.S. Cl. 424/277.1; 435/7.23; 435/69.1; 435/320.1; 435/325; 530/350; 536/23.5

(57) ABSTRACT

Disclosed are recombinant multiple epitope polypeptides (MEPs) consisting of T cell epitopes derived from tumor-associated antigens capable of being presented by an antigen presenting cell (APC), the recombinant nucleic acid sequences and expression vectors encoding them, and host cells transfected with said expression vectors. Further described are LTB-MEP fusion proteins comprising the E. Coli heat labile enterotoxin subunit B (LTB) peptide fused to a MEP by a synthetic linker, the recombinant nucleic acid sequences and expression vectors encoding them and host cell transfected with said expression vectors. Further included are compositions for inducing an immune response against malignancies, pharmaceutical compositions and a transdermal drug delivery system for the treatment of malignant disorders. Also disclosed are methods for conferring immunity against malignancies and for the treatment of malignant disorders.

### CROSS-REFERENCE TO RELATED APPLICATION

This application is a continuation-in-part of International Application No. PCT/IL2004/000894 filed Sep. 26, 2004. The contents of the related application are herein incorporated by reference in their entirety.

### FIELD OF THE INVENTION

The present invention relates to methods and compositions for conferring immunity against a malignancy in a mammalian subject. More particularly, the invention relates to a multiepitope polypeptide (MEP) capable of being presented by an antigen-presenting cell (APC) in context of MHC Class I and Class II molecules and inducing T cell activation. The invention further relates to nucleic acid sequences and constructs encoding said MEP, methods, compositions and use of said MEP in the treatment of malignant disorders.

### BACKGROUND OF THE INVENTION

The improved molecular understanding of immune recognition and regulation provides new strategies for anti-tumor vaccination. Progress in tumor immunotherapy development is apparent in lymphomas, melanomas, renal cell carcinomas and other immunogenic tumors. However, in solid tumors, the most common variety of cancer tumors, the success of clinical immunotherapy has been limited. The failure to develop effective immunity against solid tumors is due to several factors. In addition to the fact that the immune system is adversely affected by tumorgenesis, even early in the course of the disease, chemotherapeutic agents act as immunosuppressant. Since tumor antigens are usually self derived antigens, the immune response generated is often weak and with no clinical importance. In order to overcome self-tolerance and induce an immune response towards tumor antigens, the use of dendritic cells (DC) is being explored.

Recently, new approaches to immunotherapy have used antigen-loaded autologous DC in order to induce specific T-cell responses [reviewed by Sprinzl, G. M. et al., Cancer Treat. Rev. 27:247-5 (2001) and Brossart, P. et al., Exper. Hematol. 29:1247-55 (2001)]. DC which are very potent antigen presenting cells (APC), are obtained from peripheral blood monocytes of patients and cultivated ex vivo in the presence of cytokines, loaded with antigen, and returned to patients. Immunogenic tumors were first explored, with the goal of generating strong and long-lasting tumor-specific T-cell immunity. Several tumor-associated antigens (TAA) have been already defined. Cytotoxic T lymophocytes (CTL) from cancer patients are able to recognized such antigens in association with HLA class I molecules. Based on these facts, different peptide-based vaccination protocols have been developed for the treatment of various solid tumors. Protocols which use autologous DC loaded with single defined peptides, are ongoing intensive investigation in several institutions.

There is increasing consensus among researchers that DC provide strong stimuli for the generation of immunity, and that dendritic cell therapy has great potential and should be pursued aggressively [Carson, III W. E. and Khleif, S. (Chairs). Tumor vaccines, Program and abstracts of the American Society of Clinical Oncology 36^{th} Annual Meeting; May 20-23; 2000; New Orleans, La. Oral presentation]. Early pilot studies have established the safety and feasibility of this approach. In addition, these studies have demonstrated the ability of DC to produce an immunological response, although such response was less effective where it was directed towards tumors. The immunity obtained using this approach may be short lived, and still, there is no consensus with respect to the identity and format of the antigens, the type of adjuvant or other enhancers that should be used, and the optimal mode of immunization. There is a requirement to develop new immunotherapy approaches.

A number of TAAs capable of activating CTL responses and directing target cell lysis have been identified. DC can be pulsed with synthetic peptides derived from known TAAs such as MUC1, MUC2 [Bohm, C. M. et al., Int. J. Cancer 75:688-93 (1998)], Her-2/neu, CEA, PSA and others. In breast and ovarian cancer patients the use of HER-2 [Brossart, P. et al., Blood 96 (9): 3102-08 (2000); Buschenfelde, C. M. et al., J. Immunol. 167:1712-9 (2001)] and MUC1 [Brossart (2000) ibid.] were studied. These studies detected specific CTLs in peripheral blood of patients as measured by intracellular IFN-γ staining and ⁵¹Cr-release assay. Although clearly the treatment elicited specific immune responses to the peptides, clinical response was only observed in a relatively small number of patients. The most impressive results were reported lately [Fong, L. et al. Proc. Natl. Acad. Sci. 98 (15):8809-14 (2001)] in a study conducted in colorectal (n=10) and lung cancer (n=2) patients. DC were pulsed with carcinoembryonic antigen-derived peptide and then transfused to patients. Two complete responses, 1 partial response and 2 stable diseases were observed in this group.

Multiepitope Vaccines

In general, it has been shown that peptides are more efficient for antigen presentation than proteins. However, in the context of vaccination, peptides have the disadvantage of being presented exogenously, and only for a relatively short period (usually several hours). Proteins or polypeptides, on the other hand, are presented endogenously, in a more stable and long-lasting manner. Thus, an “ideal” antigen would consist of antigenic peptides, delivered as a larger molecule to be presented endogenously. Multiepitope polypeptides (MEPs) represent an attempt to produce such molecule. One major concern when generating MEPs is that they should be efficiently processed by the immunoproteasome, to enable proper presentation of the individual epitopes. Thus, the multiepitope polypeptide needs to be designed in such a way that it will include signals for proteasomal cleavage in the boundaries or linkers between the individual epitopes. Previously the inventors have shown, based on an analysis of about 300 naturally processed peptides, that both the C-terminal residue and its flanking residue play a role in determination of proteasome cleavage specificity [Altuvia, Y. and Margalit, H. J. Mol. Biol. 295:879-90 (2000)]. Furthermore, it was demonstrated recently that changing the C-terminal flanking residue could enhance immunogenicity of a multiepitope peptide [Livingston, B. D. Vaccine 19:4652-60 (2001)]. The inventors have therefore designed and produced as described by the present application, a MEP for melanoma (designated MEP-mel), which contains four immunogenic peptides from three melanoma associated proteins. As shown by the present invention, strong cytotoxic T cell responses were induced in peptide-specific T-cell clones upon transfection of dendritic cells with the MEP-Mel DNA. Presentation was sustained for at least 72 hours, and was far superior to peptide pulsing. Moreover, as shown by the present invention, transfection of DC with the whole tumor protein antigens was far less efficient, since these cells were unable to present class-I restricted epitopes derived of these antigens.

It is therefore one object of the present invention to apply a bioinformatic analysis to design multiepitope polypeptides for the treatment of malignant disorders. Such molecules may be of potential benefit for the treatment of different epithelial cancer, such as breast and ovarian carcinomas.

Another particular object of the invention is to provide specific multiepitope polypeptides comprising melanoma or breast cancer epitopes and thereby develop a novel therapeutic approach for such malignancies.

It should be noted that the multiepitope polypeptides of the invention may be applied using ex vivo loaded DC, either with DNA, RNA or protein or using carrier molecules which also act as adjuvants, for the direct delivery of the antigenic molecules to DC and to other antigen presenting cells in vivo.

It is an object of the present invention to provide a new approach for the treatment against tumors combining the novel multiepitope polypeptide of the invention with a unique transdermal drug delivery system, which is based on the use of the B subunuit of the LT protein, LTB. These and other objects of the invention will become apparent as the description proceeds.

### SUMMARY OF THE INVENTION

In a first aspect, the invention relates to a recombinant nucleic acid sequence encoding a multiepitope polypeptide (MEP) which epitopes are presented by an antigen presenting cell (APC) in the context of MHC Class I molecules. The nucleic acid sequence of the invention comprises at least two segments, wherein each of said segments, which may be identical or different, encodes a T cell epitope derived from a tumor associated antigen (TAA) and each of said segments is operably linked to an adjacent segment by a spacer element. Each of these spacer elements, which may be identical or different, encodes a signal for proteasomal cleavage.

In one specific embodiment, the recombinant nucleic acid sequence of the invention may further comprise operably linked linkers, nucleic acid segments encoding restriction enzyme sites and nucleic acid segments encoding adjuvants or carriers. Addition of specific carriers to the MEP polypeptide encoded by such nucleic acid sequence, further following proteasomal cleavage, grant presentation of MEP cleavage products by an APC in the context of MHC Class I and Class II molecules. Preferably, such carrier may be any one of enterotoxin, in particular the E. Coli heat labile enterotoxin subunit B (LTB) and Immunoglobulin Fc fragment, which enable presentation of said MEP by an APC in the context of MHC Class II molecules.

In another embodiment, the recombinant nucleic acid sequence of the invention may be any one of DNA, RNA and any combination thereof.

In a preferred embodiment, the T cell epitope encoding segments comprised within the recombinant nucleic acid sequence of the invention may be derived from TAAs which are associated with any one of carcinomas, lymphomas, melanomas and sarcomas.

In yet another embodiment, the signals for proteasomal cleavage encoded by said spacer elements comprised within the recombinant nucleic acid sequence of the invention, direct intracellular proteasomal degradation of the MEP into epitope peptides that are presented by an antigen presenting cell (APC) in the context of MHC Class I molecules.

More particularly, the signals for proteasomal cleavage encoded by these spacer elements are peptides having the amino acid sequence of any one of RKSY, RKSYL, ALL, SSL, AAY, AVHV, RVTIL and AASRY substantially as denoted by any one of SEQ ID NO: 19 to 23 and 41-43, respectively.

In one specific embodiment, the epitopes encoded by the nucleic acid sequence of the invention may be derived from any of the melanoma-associated antigens (MAA), tyrosinase, gp-100, MAGE-3 and MART-1. More particularly, these epitopes may be selected from the group consisting of peptide 280-288 of gp100, peptide 209-217 of gp100, peptide 369-377 of tyrosinase and peptide 27-35 of MART-1, substantially as denoted by the amino acid sequences as denoted by SEQ ID NO: 15 to 18, respectively, or by any functional homologue, variant, equivalent and derivative thereof.

A particular example of a recombinant nucleic acid sequence according to the invention encodes a multiepitope polypeptide (MEP) comprising at least two segments, which may be identical or different, each of these segments encodes T cell epitopes derived from melanoma associated antigens selected from the group consisting of amino acids 280-288 of gp100, amino acids 209-217 of gp100, amino acids 369-377 of tyrosinase and amino acids 27-35 of MART-1, substantially as denoted by the amino acid sequences as denoted by SEQ ID NO: 15 to 18, respectively or any homologue, variant and derivative thereof. Each of these segments may be operably linked to an adjacent segment by a spacer element, wherein each of said spacer elements, which may be identical or different, encodes a signal for proteasomal cleavage selected from the group consisting of RKSY, RKSYL, ALL, SSL AAY, substantially as denoted by SEQ ID NO: 19 to 23, respectively.

In a particular embodiment of said example, the sequence may be denoted by SEQ ID NO: 1 and encodes a melanoma-derived multiepitope polypeptide, designated MEP-Mel having the amino acid sequence as denoted by SEQ ID NO: 2, or SEQ ID NO: 57 or any functional homologue, variant, derivative and equivalent thereof.

In yet another example, the epitopes encoded by the nucleic acid sequence of the invention may derive from any of the breast and ovarian carcinoma-associated antigens, Mucin-1 (MUC1) and Lactadherin (BA46).

Such epitopes may be selected from the group consisting of peptides D6 (LLLTVLTVV) and A7 (NLTISDVSV) of MUC1, and peptides BA46-6 (NLFETPVEA) and BA46-7 (GLQHWVPEL) of Lactadherin, substantially as denoted by the amino acid sequences as denoted by SEQ ID NO: 25 to 28, respectively, or any functional homologue, derivative, variant and equivalent thereof.

A particular embodiment of a nucleic acid sequence encoding an epithelial carcinoma derived MEP, may therefore comprise at least two segments, which may be identical or different, wherein each of said segments encodes epitopes derived from breast and ovarian carcinoma-associated antigens selected from the group consisting of peptides D6 (LLLTVLTVV) and A7 (NLTISDVSV) of MUC1, peptides BA46-6 (NLFETPVEA) and BA46-7 (GLQHWVPEL) of Lactadherin, substantially as denoted by the amino acid sequences as denoted by SEQ ID NO: 25 to 28, respectively. Each of these segments may be operably linked to an adjacent segment by a spacer element. Each of said spacer elements, which may be identical or different, encodes a signal for proteasomal cleavage selected from the group consisting of: RKSYL, AAY, AVHV, RVTIL and AASRY substantially as denoted by SEQ ID NO: 20, 23 and 41 to 43, respectively.

Such specific nucleic acid sequence may therefore be a sequence denoted by SEQ ID NO: 39 and encoding a breast cancer derived multiepitope polypeptide, designated MEP-Epi, having the amino acid sequence as denoted by SEQ ID NO: 40, or any functional homologue, derivative, variant and equivalent thereof.

It should be noted that any of the specific nucleic acid sequences of the invention may further comprise operably linked linkers, nucleic acid segments encoding restriction enzyme sites and nucleic acid segments encoding adjuvants or carriers. Such nucleic acid sequence may encode a MEP which epitopes are presented by an APC in the context of MHC Class I and Class II molecules. In a particular embodiment, such carrier may be any one of enterotoxin, preferably E. Coli heat labile enterotoxin subunit B (LTB) and Immunoglobulin Fc fragment.

In a second aspect, the present invention relates to an expression vector encoding a multiepitope polypeptide which epitopes are presented by an antigen-presenting cell (APC) in the context of MHC Class I molecules. Such expression vector according to the invention may comprise (a) a recombinant nucleic acid sequence encoding a multiepitope polypeptide (MEP) as defined by the invention. This sequence comprises at least two segments, wherein each of said segments, which may be identical or different, encodes a T cell epitope derived from a tumor associated antigen (TAA) and wherein each of said segments is operably linked to an adjacent segment by a spacer element, wherein each of said spacer elements, which may be identical or different, encodes a signal for proteasomal cleavage; and (b) operably linked control, promoting and/or regulatory elements.

In one embodiment, the recombinant nucleic acid sequence comprised within the expression vector of the invention may further comprise operably linked linkers, nucleic acid segments encoding restriction enzyme sites and nucleic acid segments encoding adjuvants or carriers. Such nucleic acid sequence encodes a MEP, wherein following said proteasomal cleavage, cleavage products of said MEP are presented by an APC in the context of MHC Class I and Class II molecules.

According to a specifically preferred embodiment, the expression vector of the invention may be a DNA or RNA expression vector.

In yet another embodiment, the expression vectors of the invention may comprise any of the nucleic acid sequences as defined by the invention.

The invention further provides for a host cell transfected with any of the expression vectors defined by the invention.

The host cell of the invention may be any one of eukaryotic and prokaryotic cell.

In a particular embodiment, the expression vector host cell may be a mammalian cell. A specifically preferred mammalian host cell may be an antigen-presenting cell (APC) selected from dendritic cells (DC), activated B cells, and activated macrophages.

A third aspect of the invention relates to a recombinant multiepitope polypeptide (MEP) capable of being presented by an antigen presenting cell (APC) in the context of MHC Class I molecules and inducing T cell activation. The MEP of the invention may comprise at least two T cell epitopes (antigenic determinant) derived from a tumor-associated antigen (TAA), which may be identical or different. Each of these epitopes may be operably linked to an adjacent epitope by a signal for proteasomal cleavage. It should be noted that these signals might be identical or different.

In a specific embodiment, the MEP of the invention may further comprise adjuvants or carriers. Such addition of particular carrier enables presentation of the MEP of the invention by an APC, in the context of MHC Class I and Class II molecules. In a particular embodiment, such carrier may be enterotoxin, preferably E. Coli heat labile enterotoxin subunit B (LTB) or Immunoglobulin Fc fragment.

According to a specifically preferred embodiment, the epitopes comprised within the MEP of the invention are derived from TAAs, which are associated with any one of carcinomas, lymphomas, melanomas and sarcomas.

In yet another specifically preferred embodiment, the signals for proteasomal cleavage comprised within the MEP of the invention, direct intracellular proteasomal degradation of said MEP into epitope and/or peptide fragments presented by an antigen presenting cell (APC) in the context of MHC Class I and Class II molecules. The signals for proteasomal cleavage may be according to preferred embodiment, peptides selected from the group consisting of RKSY, RKSYL, ALL, SSL, AAY, AVHV, RVTIL and AASRY substantially as denoted by any one of the amino acid sequences of SEQ ID NO: 19 to 23 and 41 to 43, respectively.

In one specific embodiment, the epitopes comprised within the MEP of the invention are derived from any of the melanoma-associated antigens (MAA) tyrosinase, gp-100, MAGE-3 and MART-1.

Preferably, these epitopes may be selected from the group consisting of peptides 280-288 and 209-217 of gp100, peptide 369-377 of tyrosinase and peptide 27-35 of MART-1, as denoted by the amino acid sequences as denoted by SEQ ID NO: 15 to 18, respectively, or by any functional homologue, derivative, variant and equivalent thereof

Therefore, a recombinant multiepitope polypeptide (MEP) of the invention may be for example, a MEP comprising at least two T cell epitopes, which may be identical or different, derived from melanoma-associated antigens selected from the group consisting of amino acids 280-288 of gp100, amino acids 209-217 of gp100, amino acids 369-377 of tyrosinase and amino acids 27-35 of MART-1, substantially as denoted by the amino acid sequences as denoted by SEQ ID NO: 15 to 18, respectively. Each of said epitopes is linked to an adjacent epitope by a signal for proteasomal cleavage. Such signals, which may be identical or different, may be selected from the group consisting of RKSY, RKSYL, ALL, SSL AAY, substantially as denoted by SEQ ID NO: 19 to 23, respectively.

A particular example of a melanoma-derived multiepitope polypeptide is the MEP designated MEP-MEL, which has the amino acid sequence as denoted by SEQ ID NO: 2 or SEQ ID NO: 57 or any functional homologue, derivative, variant and equivalent thereof.

In another specific embodiment, the epitopes comprised within the MEP molecule of the invention may be derived from the breast and ovarian carcinoma associated antigens, Mucin-1 (MUC1) and Lactadherin (BA46).

In a preferred embodiment, such recombinant polypeptide may comprise epitopes selected from the group consisting of peptides D6 (LLLTVLTVV) and A7 (NLTISDVSV) of MUC1, and peptides BA46-6 (NLFETPVEA) and BA46-7 (GLQHWVPEL) of Lactadherin, substantially as denoted by the amino acid sequences as denoted by SEQ ID NO: 25 to 28, respectively, or by any functional homologue, derivative, variant and equivalent thereof.

Accordingly, the invention therefore provides for a recombinant multiepitope polypeptide (MEP) comprising at least two T cell epitopes derived from epithelial carcinoma-associated antigens. These epitopes may be identical or different and may be selected from the group consisting of peptides D6 (LLLTVLTVV) and A7 (NLTISDVSV) of MUC1, peptides BA46-6 (NLFETPVEA) and BA46-7 (GLQHWVPEL) of Lactadherin, as denoted by the amino acid sequences of SEQ ID NO: 25 to 28, respectively. Each of said epitopes is linked to an adjacent epitope by a signal for proteasomal cleavage that may be identical or different. Such signal may be selected from the group consisting of RKSYL, AAY, AVHV, RVTIL and AASRY substantially as denoted by SEQ ID NO: 20, 23 and 41 to 43, respectively.

A particular example for such recombinant polypeptide, is the epithelial MEP designated MEP-Epi having the amino acid sequence as denoted by SEQ ID NO: 40, or by any functional homologue, derivative, variant and equivalent thereof.

It should be noted that any of the recombinant polypeptides of the invention may be produced synthetically or preferably, may be produced by any of the host cells as defined by the invention.

Another aspect of the invention relates to a recombinant nucleic acid encoding a fusion protein (LTB-MEP) composed of a MEP sequence linked via a suitable linking element to the nucleic acid sequence encoding LTB. LTB-MEP segments and/or fragments resulting from proteasomal cleavage are presented by an antigen presenting cell (APC) in the context of MHC Class I and Class II molecules.

This recombinant nucleic acid sequence encoding a fusion protein combines the LTB sequence to a multiepitope polypeptide (MEP) which sequence comprises at least two segments, wherein each of said segments, which may be identical or different, encodes a T cell epitope derived from a tumor associated antigen (TAA), and wherein each of said segments is operably linked to an adjacent segment by a spacer element, wherein each of said spacer elements, which may be identical or different, encodes a signal for proteasomal cleavage

In another embodiment, the recombinant nucleic acid sequence encoding an LTB-MEP fusion protein of the invention may be any one of DNA, RNA and any combination thereof.

In a preferred embodiment, the T cell epitope encoding segments comprised within the recombinant nucleic acid sequence of the LTB-MEP fusion protein in the invention may be derived from TAAs which are associated with any one of carcinomas, lymphomas, melanomas and sarcomas.

In one specific embodiment, the epitopes encoded by the fusion protein recombinant nucleic acid sequence of the invention may be derived from any of the melanoma-associated antigens (MAA), tyrosinase, gp-100, MAGE-3 and MART-1. More particularly, these epitopes may be selected from the group consisting of peptide 280-288 of gp100, peptide 209-217 of gp100, peptide 369-377 of tyrosinase and peptide 27-35 of MART-1, substantially as denoted by the amino acid sequences as denoted by SEQ ID NO: 15 to 18, respectively, or by any functional homologue, variant, equivalent and derivative thereof.

A particular example of a recombinant nucleic acid sequence encoding for the fused protein according to the invention, comprises a multiepitope polypeptide (MEP) consisting of at least two segments, which may be identical or different, each of these segments encodes T cell epitopes derived from melanoma associated antigens selected from the group consisting of amino acids 280-288 of gp100, amino acids 209-217 of gp100, amino acids 369-377 of tyrosinase and amino acids 27-35 of MART-1, substantially as denoted by the amino acid sequences as denoted by SEQ ID NO: 15 to 18, respectively or any homologue, variant and derivative thereof. Each of these segments may be operably linked to an adjacent segment by a spacer element, wherein each of said spacer elements, which may be identical or different, encodes a signal for proteasomal cleavage selected from the group consisting of RKSY, RKSYL, ALL, SSL AAY, substantially as denoted by SEQ ID NO: 19 to 23, respectively.

In a particular embodiment of said example, the sequence may be denoted by SEQ ID NO: 54 and encodes a melanoma-derived multiepitope polypeptide-LTB fused protein, designated LTB-MEP-Mel having the amino acid sequence as denoted by SEQ ID NO: 56, or any functional homologue, variant, derivative and equivalent thereof.

In yet another example, the epitopes encoded by the recombinant nucleic acid fusion protein LTB-MEP of the invention may derive from any of the breast and ovarian carcinoma-associated antigens, Mucin-1 (MUC1) and Lactadherin (BA46).

Such epitopes may be selected from the group consisting of peptides D6 (LLLTVLTVV) and A7 (NLTISDVSV) of MUC1, and peptides BA46-6 (NLFETPVEA) and BA46-7 (GLQHWVPEL) of Lactadherin, substantially as denoted by the amino acid sequences as denoted by SEQ ID NO: 25 to 28, respectively, or any functional homologue, derivative, variant and equivalent thereof.

A particular embodiment of a nucleic acid sequence encoding an epithelial carcinoma derived MEP in the fused protein, may therefore comprise at least two segments, which may be identical or different, wherein each of said segments encodes epitopes derived from breast and ovarian carcinoma-associated antigens selected from the group consisting of peptides D6 (LLLTVLTVV) and A7 (NLTISDVSV) of MUC1, peptides BA46-6 (NLFETPVEA) and BA46-7 (GLQHWVPEL) of Lactadherin, substantially as denoted by the amino acid sequences as denoted by SEQ ID NO: 25 to 28, respectively. Each of these segments may be operably linked to an adjacent segment by a spacer element. Each of said spacer elements, which may be identical or different, encodes a signal for proteasomal cleavage selected from the group consisting of: RKSYL, AAY, AVHV, RVTIL and AASRY substantially as denoted by SEQ ID NO: 20, 23 and 41 to 43, respectively.

In a particular embodiment of said example, the sequence that encodes a melanoma-derived multiepitope polypeptide fused to LTB is designated LTB-MEP-Mel composed by the LTB sequence as denoted by SEQ ID NO: 52, linked to the nucleic acid sequence encoding a breast cancer derived multiepitope polypeptide denoted by SEQ ID NO: 39 or any functional homologues, derivatives, variants and equivalents thereof.

In a broad aspect, the present invention relates to an expression vector encoding a multiepitope fusion protein (LTB-MEP) composed of a MEP sequence linked via a suitable linking element to the nucleic acid sequence encoding LTB which segments and/or fragments resulting from the proteasomal cleavage are presented by an antigen presenting cell (APC) in the context of MHC Class I and Class II molecules.

Such expression vector according to the invention may comprise (a) a nucleic acid sequence encoding for a multiepitope polypeptide (MEP) which comprises at least two segments, wherein each of said segments, which may be identical or different, encodes a T cell epitope derived from a tumor associated antigen (TAA), and wherein each of said segments is operably linked to an adjacent segment by a spacer element, wherein each of said spacer elements, which may be identical or different, encodes a signal for proteasomal cleavage, operably linked via a suitable linking element, to (b) a nucleic acid sequence encoding LTB, and operably linked to (c) control, promoting and/or regulatory elements.

In an extended embodiment, the recombinant nucleic acid sequence comprised within the LTB-MEP fusion protein expression vector may include any of the MEP nucleic acid sequences described in the invention linked to LTB sequences.

In another embodiment, the recombinant nucleic acid sequence comprised within the LTB-MEP fusion protein expression vector of the invention may further comprise operably linked linkers, nucleic acid segments encoding restriction enzyme sites and nucleic acid segments encoding adjuvants or carriers.

According to a specifically preferred embodiment, the LTB-MEP fusion protein expression vector of the invention may be a DNA or RNA expression vector.

The invention also includes an expression system consisting of a host cell transfected with any one of the LTB-MEP fusion protein expression vectors mentioned above, wherein said host cell is any one of eukaryotic and prokaryotic cell, preferably a mammalian antigen presenting cell (APC).

In a different aspect, the invention relates to a fusion protein comprising the LTB peptide sequence fused to a MEP polypeptide (LTB-MEP), encoded by any of the LTB-MEP fusion protein expression vectors. LTB-MEP cleavage products (epitopes and fragments) of said fusion protein are presented by an APC in the context of MHC Class I and Class II molecules and induce B and T cell activation.

In a particular embodiment of the invention, the fusion protein comprises a melanoma-associated multiepitope polypeptide fused to LTB. This fusion protein is designated LTB-MEP-Mel, and it is defined by the amino acid sequence as denoted by SEQ ID NO: 56, or by any functional analogue, variant, equivalent and derivative thereof

Another fusion protein may comprise an epithelial carcinoma derived multiepitope, for example an LTB-breast cancer derived multiepitope fusion protein composed of SEQ ID NOs: 52, 53 and 39, or any functional analogue, variant, equivalent and derivative thereof, designated LTB-MEP-Epi

In a further aspect, the invention relates to a composition for inducing an immune response directed against malignancy in a mammalian subject. The compositions of the invention may comprise at least one active ingredient chosen from the multiepitope polypeptide (MEP) and/or LTB-MEP fusion protein of the invention, a recombinant nucleic acid sequence and expression vector encoding said multiepitope polypeptide (MEP) or LTB-MEP fusion protein, and a host cell transfected with said vectors as defined by the invention.

The invention further provides for a pharmaceutical composition for the treatment of a malignant disorder in a mammalian subject. The pharmaceutical composition of the invention may comprise as an active ingredient a multiepitope polypeptide (MEP) or an LTB-MEP fusion protein of the invention, a recombinant nucleic acid sequence and an expression vector encoding said multiepitope polypeptide (MEP) or LTB-MEP fusion protein, or a host cell transfected with said vectors as defined by the invention.

The compositions of the invention may optionally further comprise pharmaceutically acceptable carrier, diluent, excipient and additive.

In a specific embodiment, the compositions of the invention may further comprise an IgG Fc fragment and enterotoxin, conjugated to or mixed with said active ingredient.

According to a preferred embodiment, the compositions are intended for use in malignancies or malignant disorders such as carcinomas, lymphomas, melanomas and sarcomas.

In a particular embodiment, the compositions of the invention may be used where the malignant disorder is melanoma.

The compositions of the invention are particularly intended for use in a mammalian subject such as human.

In yet another preferred embodiment, the APC comprised as an active ingredient, within the composition of the invention, may be autologous dendritic cells (DC). It should be noted that these cells may be transfected with the nucleic acid sequence or the expression vectors of the invention, or alternatively, may be loaded with the MEP of the invention.

Another aspect of the invention relates to a method for conferring immunity against a malignancy in a mammalian subject, comprising the steps of administering to a subject in need, the multiepitope polypeptide (MEP) or LTB-MEP fusion protein of the invention, a recombinant nucleic acid sequence or an expression vector encoding said MEP or LTB-MEP fusion protein, a host cell transfected with said vectors, an autologous APC loaded with said MEP or LTB-MEP fusion protein, or a composition comprising the same as defined by the invention, in an amount sufficient to induce an immune response against said malignancy in a subject in need.

The invention further provides for a method for the treatment of a malignant disorder in a mammalian subject in need. This method comprises the step of administering to said subject the multiepitope polypeptide (MEP) or LTB-MEP fusion protein of the invention, a recombinant nucleic acid sequence or an expression vector encoding said MEP or LTB-MEP fusion protein, a host cell transfected with said vectors, an autologous APC loaded with said MEP or LTB-MEP fusion protein, or a composition comprising the same as defined by the invention, in an amount sufficient to induce in said subject an immune response against said malignancy.

The method of the invention may be applicable for malignancies such as carcinomas, lymphomas, melanomas and sarcomas.

In a particular embodiment, the methods of the invention are applicable where the malignancy or malignant disorder is melanoma.

In yet another embodiment, the methods of the invention are intended for the treatment of a mammal, preferably, a human.

According to a particular embodiment, where host cell transfected with the vectors of the invention are administered to a subject in need according to the method of the invention, such cells may be preferably autologous APCs. Most preferably, autologous dendritic cell (DC).

Another aspect of the invention refers to a drug delivery, particularly, but not limited to a transdermal delivery system for the treatment of a malignant disorder. This transdermal drug delivery system induces a systemic antigen specific immune response in a mammalian subject in need. Said immune response results in the production of antibodies, helper and cytotoxic T lymphocytes, specific for different antigens associated with said malignancy, comprised in said LTB-MEP fusion protein.

The system comprises as an active ingredient at least the B subunit of the LT protein (LTB) conjugated to or mixed with anyone of the multiepitope polypeptide MEP described in the invention, optionally further comprises pharmaceutically acceptable carrier, diluent, excipient, adjuvant and/or additive.

In a particular embodiment, the transdermal drug delivery system comprises said LTB recombinantly fused to MEP, creating any of the LTB-MEP fusion proteins described in the invention.

The transdermal delivery system is applied in the form of an ointment, cream, spray, patches, sustained-release patches, osmotic pumps or any other suitable transdermal delivery vehicle.

In a broad aspect, the transdermal drug delivery system is used for the treatment of a malignancy selected from carcinomas, lymphomas, melanomas and sarcomas groups.

In a specific embodiment, the transdermal drug delivery system is used for the treatment of melanoma.

Finally, the invention embraces a method for the treatment of a malignant disorder in a mammalian subject in need, by applying to said subject the transdermal drug delivery system of the invention, in an amount sufficient to induce in said subject a systemic immune response against said malignancy. This treatment method induces an immune response which results in the production of antibodies, helper and cytotoxic T lymphocytes, specific for different antigens associated with said malignancy, comprised in said LTB-MEP fusion protein.

In one embodiment, said method is for the treatment of a malignancy, selected from the group consisting of carcinomas, lymphomas, melanomas and sarcomas.

In a preferably embodiment, said method should be used for the treatment of melanoma.

In yet another embodiment, this treatment method of the invention is intended for the treatment of a mammal, preferably, a human.

It should be appreciated that the immune response initiated by any of the methods of the invention results in a cellular (involves T cells) and/or humoral (B cells mediated) response. This response may induce the production of both helper and cytotoxic T lymphocytes, as well as antibodies specific for different antigens associated with said malignancy which are comprised in the MEP or LTB-MEP fusion proteins of the invention. Said immune response should be systemic, although the treatment is provided by of local application.

Still further, the invention relates to the use of a multiepitope polypeptide (MEP) and/or an LTB-MEP fusion protein capable of being presented by an antigen presenting cell (APC) in the context of any one of MHC Class I and Class II, in the preparation of a pharmaceutical composition defined by the invention and a transdermal drug delivery system, for the treatment of a malignant disorder.

### BRIEF DESCRIPTION OF THE FIGURES

FIG. 1A-1B Expression of the MEP-Mel recombinant protein

FIG. 1A: Shows coommassie blue staining of an SDS-PAGE containing different fractions of the isolated proteins.

FIG. 1B: Shows an immunoblot using anti-histidine antibodies for identification of recombinant MEP-Mel protein. As negative control, pellet of proteins of bacteria carrying wild type plasmid was used (lane 1); size marker (lane 2); pellet treated with 4M urea (lane 3); supernatant treated with 4M urea (lane 4); pellet treated with 6M urea (lane 5); supernatant treated with 6M urea (lane 6); as another negative control, supernatant of proteins of bacteria carrying wild type plasmid treated with 8M urea was used (lane 7).

FIG. 2 DC presentation of MEP protein to T cell clones

DC incubated with MEP-Mel protein were able to specifically stimulate T cell clones reactive to different MEP epitopes of gp100 and MART-1. T cell stimulation was estimated by the amount of IFN-γ secreted to the culture medium (pg/ml).

T cell clones tested: GP 100-209a and 209b specific for gp100 209-217 epitope, GP 100-280 specific for gp100 280-288 epitope, GP 100-154 specific for a gp100 epitope not present in MEP (used as control) and MART-1 specific for the epitope 27-35.

Abbreviations: :Cl. Sp.: T cell clone specificity; IFN-γ: interferon gamma.

FIG. 3 Human skin incubated with FITC-labeled LTB-MEP

Confocal microscopy picture of human epidermis previously incubated with FITC-labeled LTB-MEP. FITC labeling was observed inside cells resembling Langerhans cell morphology.

Abbreviations: FITC: flourescein isothiocyanate; LTB: Escherichia coli heat-labile enterotoxin B subunit; MEP: multiple epitope polypeptide.

FIG. 4 Anti-LTB-MEP antibody production

Anti-LTB-MEP antibodies detection in serum of mice treated with topical LTB-MEP, boiled LTB-MEP or PBS.

Abbreviations: OD: optical density; LTB: Escherichia coli heat-labile enterotoxin B subunit; MEP: multiple epitope protein.

### DETAILED DESCRIPTION OF THE INVENTION

In a first aspect, the invention relates to a recombinant nucleic acid sequence encoding a multiepitope polypeptide (MEP) which sequence comprises at least two segments, wherein each of said segments, which may be identical or different, encodes a T cell epitope derived from a tumor associated antigen (TAA), which epitope s presented by an antigen presenting cell (APC) in the context of MHC Class I molecules, and each of said segments is operably linked to an adjacent segment by a spacer element. Each of these spacer elements, which may be identical or different, encodes a signal for proteasomal cleavage.

As used herein, the term “nucleic acid” refers to polynucleotides such as deoxyribonucleic acid (DNA), and, where appropriate, ribonucleic acid (RNA). The terms should also be understood to include, as equivalents, analogues of either RNA or DNA made from nucleotide analogues, and, as applicable to the embodiment being described, single-stranded and double-stranded polynucleotides. Recombinant nucleic acid sequence is a molecule made of segments, which are naturally not normally linked in the same manner. Thus, the recombinant MEP of the invention is a continuous nucleic acid molecule having sequences operably linked, typically translated to a single product that exhibits properties derived from the original segments.

The term “epitope” is defined as the minimal structural unit of an antigen, recognizable for antibodies and lymphocyte antigenic receptors, that comes in contact with the antigen binding site of an antibody or the T-cell receptor. Epitope is meant to refer to a molecular region on the surface of an antigen capable of eliciting specific immune responses, such as the production of antibodies or activation of immune cells, and of combining with the specific antibody produced by such a response.

Epitopes or “antigenic determinants” usually consist of chemically active surface groupings of molecules such as amino acids or sugar side chains, and have specific three-dimensional structural characteristics as well as specific charge characteristics. The epitopes encoded by the nucleic acid sequences of the invention may preferably derive from tumor associated antigens. An “antigen” is a molecule or a portion of a molecule capable of being bound by an antibody, and which is additionally capable of inducing an animal to produce antibody capable of binding to an epitope of that antigen. An antigen may have one, or more epitopes.

In one specific embodiment, the recombinant nucleic acid sequence of the invention may further comprise operably linked linkers, nucleic acid segments encoding restriction enzyme sites and nucleic acid segments encoding adjuvants or carriers. Addition of specific carriers to the MEP polypeptide encoded by such nucleic acid sequence may enable presentation of MEP by an APC in the context of any one of MHC Class I and Class II molecules. Preferably, such carrier may be any one of enterotoxin and Immunoglobulin Fc fragment, which enables presentation of said MEP by an APC in the context of MHC Class II molecules.

In another embodiment, as indicated above, the recombinant nucleic acid sequence of the invention may be any one of DNA, RNA and any combination thereof.

In a preferred embodiment, the T cell epitope encoding segments comprised within the recombinant nucleic acid sequence of the invention may be derived from TAAs, which are associated with any one of carcinomas, lymphomas, melanomas and sarcomas.

In yet another embodiment, the signals for proteasomal cleavage encoded by said spacer elements comprised within the recombinant nucleic acid sequence of the invention, direct intracellular proteasomal splitting of the MEP into epitope peptides which can be presented by an antigen presenting cell (APC) in the context of MHC Class I molecules.

More particularly, the signals for proteasomal cleavage encoded by these spacer elements are peptides having the amino acid sequence of any one of RKSY, RKSYL, ALL, SSL, AAY, AVHV, RVTIL and AASRY substantially as denoted by any one of SEQ ID NO: 19 to 23 and 41 to 43, respectively. It should be appreciated that the invention further encompasses any appropriate proteasomal cleavage signal.

In one specific embodiment, the epitopes encoded by the nucleic acid sequence of the invention may be derived from any of the melanoma-associated antigens (MAA). Among multiple human melanoma antigens that were identified [Kawakami et al, Microbiol. Immunol. 42(12):801-13 (1998)], the melanosomal proteins gp-100, MART-1 and TRP-1 and -2 contain immunogenic epitopes restricted to HLA-A2 allele. CTLs recognizing these epitopes were isolated from peripheral blood of patients that experienced major melanoma regressions after immunotherapy [Andersen, M. H, et al. Int. J. Cancer 94(6):820-4 (2001); Dudley, M. E. et al. Cancer J. 6(2):69-77 (2000)] attesting to their role in successful tumor rejection.

More particularly, these epitopes may be selected from the group consisting of peptide 280-288 of gp100, peptide 209-217 of gp100, peptide 369-377 of tyrosinase and peptide 27-35 of MART-1, substantially as denoted by the amino acid sequences as denoted by SEQ ID NO: 15 to 18, respectively, or by any homologue, equivalent, derivative, variant and functional analogue thereof.

A particular example of a recombinant nucleic acid sequence according to the invention encodes a multiepitope polypeptide (MEP) comprising at least two segments, which may be identical or different, each of these segments encodes T cell epitopes derived from melanoma associated antigens selected from the group consisting of amino acids 280-288 of gp100, amino acids 209-217 of gp100, amino acids 369-377 of tyrosinase and amino acids 27-35 of MART-1, substantially as denoted by the amino acid sequences as denoted by SEQ ID NO: 15 to 18, respectively. Each of these segments may be operably linked to an adjacent segment by a spacer element, wherein each of said spacer elements, which may be identical or different, encodes a signal for proteasomal cleavage selected from the group consisting of RKSY, RKSYL, ALL, SSL AAY, substantially as denoted by SEQ ID NO: 19 to 23, respectively.

In a particular embodiment of said example, the sequence may be denoted by SEQ ID NO: 1 and encodes a melanoma-derived multiepitope polypeptide, designated MEP-Mel having the amino acid sequence as denoted by SEQ ID NO: 2 or SEQ ID NO: 57 or by any homologue, equivalent, derivative, variant and functional analogue thereof.

It should be noted that the nucleic acid sequence encoding the multiepitope peptide of the invention may be altered by taking advantage of the degeneracy of the genetic code to alter the coding sequence such that, while the nucleotide sequence is substantially altered, it nevertheless encodes an amino acid sequence substantially similar to the disclosed sequences.

Based upon the degeneracy of the genetic code, variant DNA molecules may be derived from the cDNA and gene sequences using standard DNA mutagenesis techniques or by synthesis of DNA sequences.

Thus, this invention also encompasses nucleic acid sequences which encode the MEP-Mel polypeptide of the invention, but which vary from the disclosed nucleic acid sequences by virtue of the degeneracy of the genetic code.

The term “within the degeneracy of the genetic code” used herein means possible usage of any nucleotide combinations as codons that code for the same amino acid. In other words, such changes in the nucleic acid sequences that are not reflected in the amino acid sequence of the encoded protein

By “derivatives”, “variants”, “analogues” or “derivatives” ” is meant the “derivatives”, “variants” or “analogues” of coding nucleic acid molecule or of amino acid sequence. A “variant” of such molecule is meant to refer to a naturally occurring molecule substantially similar to either the entire molecule or a fragment thereof. An “analogue” of a molecule can be a homologous molecule from the same species or from different species. The amino acid sequence of an analogue or derivative may differ from the original sequence, when at least one residue is deleted, inserted or substituted. Specifically, an analogue or derivative of the nucleic acid sequence or amino acid sequence of the invention may comprise at least one mutation, point mutation, nonsense mutation, missense mutation, deletion, insertion or rearrangement.

In yet another example, the epitopes encoded by the nucleic acid sequence of the invention may derive from any of the breast and ovarian carcinoma-associated antigens, Mucin-1 (MUCL) and Lactadherin (BA46).

MUC1 is a large glycosylated mucin expressed on most secretory epithelia including the mammary gland, gastrointestinal, respiratory, and reproductive ducts. It is overexpressed on more than 90% of breast and ovarian cancers and on 50-90% of other carcinomas, such as lung and stomach [reviewed by von Mensdorff-Pouilly et al, (2000) and Syrigos et al, (1999)]. Therefore, it is a suitable candidate for broadly applicable vaccine therapies. The unique extracellular domain, consisting mostly of 20-60 tandem repeats, is aberrantly glycosylated in cancer cells. Therefore, the exposed epitopes on its peptide core gain access to the circulation and induce humoral and cellular immune responses. The presence of natural antibodies to MUC1 in the circulation of patients diagnosed with early breast cancer was associated with better survival. A group headed by Lea Eisenbach [Carmon, L. et al, Int. J. Cancer 85:391-7 (2000)], has identified new breast-tumor associated MUC1-derived peptides. Using a unique mouse model of DbX(2 microglobulin null mice transgenic for a chimeric HLA-A2.1/Db-(2 microglobulin single chain (HHD mice), they showed that three peptides, MUC1/A7, MUC1/D6, and MUC1/E6 have high MHC-binding affinity, and -upon vaccination of HHD mice, induced CTL effectively lysed the human carcinoma lines in an HLA-A2.1-restricted manner. CTL induced against these 3 peptides also lysed more efficiently target cells loaded with peptide extracts of fresh human tumors as compared to target cells loaded with peptide extract from corresponding normal tissue [Carmon (2000) ibid.]. Adoptive transfer of anti-peptide CTL from HHD mice to nude mice bearing a human MDA-MB-231 breast carcinoma explant caused reduced growth of the tumor. It was also found that MUC1/A7 and MUC1/D6 could activate CTLs in blood lymphocytes of 2/4 and 3/4 breast carcinoma patients respectively (personal communications). Therefore, the inventors used two of the MUC1 peptides which induced strongest CTL response peptide D6 (LLLTVLTVV) and peptide A7 (NLTISDVSV), in the preparation of an epithelial carcinoma's specific MEP.

Lactadherin, a protein known to be expressed in breast carcinomas, has been recently shown to be also expressed in ovarian (SKOV), colon and bladder carcinomas [Carmon, L. et al., J. Clin. Invest. 110:453-62 (2002)]. Six of 7 non-small cell lung carcinoma lines and one small cell lung carcinoma tested showed overexpression of lactad herin. In addition, novel lactadherin-derived peptides have been recently described. Two of the peptides elicited specific CTL activity in mice as wells as in peripheral blood lymphocytes of breast carcinoma patients [Carmon (2002) ibid.]. These two peptides, BA46-6 (NLFETPVEA) and BA46-7 (GLQHWVPEL), were also used as epitopes in the epithelial specific MEP of the invention as described by Example 8.

The antigenic epitopes of both Lactadherin and MUC1 represent attractive antigens to be used in DC-based vaccines in patients with metastatic carcinomas with MUC1 or Lactadherin expressing tumors. Therefore, these epitopes were used for preparation of an epithelial specific MEP.

According to a specific embodiment therefore, such epitopes may be selected from the group consisting of peptides D6 (LLLTVLTVV) and A7 (NLTISDVSV) of MUC1, and peptides BA46-6 (NLFETPVEA) and BA46-7 (GLQHWVPEL) of Lactadherin, as denoted by the amino acid sequences substantially as denoted by SEQ ID NO: 25 to 28, respectively, or by any homologue, equivalent, derivative, variant and functional analogue thereof.

A particular embodiment thus relates to a nucleic acid sequence which encodes an epithelial carcinoma derived MEP. This sequence comprises at least two segments, which may be identical or different, wherein each of said segments encodes epitopes derived from breast and ovarian carcinoma-associated antigens selected from the group consisting of peptides D6 (LLLTVLTVV) and A7 (NLTISDVSV) of MUC1, peptides BA46-6 (NLFETPVEA) and BA46-7 (GLQHWVPEL) of Lactadherin, substantially as denoted by the amino acid sequences as denoted by SEQ ID NO: 25 to 28, respectively. Each of these segments may be operably linked to an adjacent segment by a spacer element. Each of said spacer elements, which may be identical or different, encodes a signal for proteasomal cleavage selected from the group consisting of: RKSYL, AAY, AVHV, RVTIL and AASRY substantially as denoted by SEQ ID NO: 20, 23, 41 to 43, respectively.

Such specific nucleic acid sequence may therefore be a sequence denoted by SEQ ID NO: 39 and encoding a breast cancer derived multiepitope polypeptide, designated MEP-Epi, having the amino acid sequence as denoted by SEQ ID NO: 40, or by any analogue, variant, equivalent, derivative and functional analogue thereof.

It should be noted that any of the specific nucleic acid sequences of the invention may further comprise operably linked linkers, nucleic acid segments encoding restriction enzyme sites and nucleic acid segments encoding adjuvants or carriers. Such nucleic acid sequence may encode a MEP wherein following proteasomal cleavage, cleavage products of said MEP are presented by an APC in the context of MHC Class I and Class II molecules.

In a particular embodiment, one preferred carrier may be human recombinant Fc fragment of immunoglobulin which, when fused with an antigen, can induce enhancement of antigen presentation and stimulation of cytotoxic and helper lymphocytes. This is based on the fact that Fc receptors are expressed on most cells of the hemopoietic lineage, including DC, and antigen uptake through this pathway can affect antigen presentation in the context of Class I and of Class II [Amigorena, et al. Semin. Immunol. 11:385 (1999,); Amigorena, et al. Immunol. Rev: 172:279 (1999)].

Another possible carrier may be derived from microbial products, which are danger signals that have been shown to activate adaptive immunity, including CD8+ T cells. E. Coli heat labile enterotoxin (LT) is such a molecule; LT and its B subunit LTB have been demonstrated as appropriate carriers and potent systemic and mucosal adjuvants for co-administered antigens. A non-toxic form of LT (NLT) was produced by the present inventors. Preliminary data indicate that NLT and LTB applied topically enter epidermal Langerhans' cells, and induce DC maturation in vitro. Thus, NLT and LTB could be developed as a delivery system for the presentation of tumor antigens to the immune system.

In a second aspect, the present invention relates to an expression vector encoding a multiepitope polypeptide which epitopes are presented by an antigen presenting cell (APC) in the context of MHC Class I molecules. Such expression vector according to the invention, may comprise (a) a recombinant nucleic acid sequence encoding a multiepitope polypeptide (MEP) as defined by the invention. This sequence comprises at least two segments, wherein each of said segments, which may be identical or different, encodes a T cell epitope derived from a tumor associated antigen (TAA) and wherein each of said segments is operably linked to an adjacent segment by a spacer element, wherein each of said spacer elements, which may be identical or different, encodes a signal for proteasomal cleavage; and (b) operably linked control, promoting and/or regulatory elements.

In one embodiment, the recombinant nucleic acid sequence comprised within the expression vector of the invention may further comprise operably linked linkers, nucleic acid segments encoding restriction enzyme sites and nucleic acid segments encoding adjuvants or carriers. Such nucleic acid sequence encodes a MEP capable of being presented by an APC in the context of any one of MHC Class I and Class II molecules.

According to a specifically preferred embodiment, the expression vector of the invention may be a DNA or RNA expression vector.

The expression vector of the invention may further comprise operably linked regulatory elements. The term “operably linked” is used herein for indicating that a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the proper reading frame.

Accordingly, the term control and regulatory elements includes promoters, terminators and other expression control elements. Such regulatory elements are described by Goeddel [Goeddel, Gene Expression Technology: Methods in Enzymology 185, Academic Press, San Diego, Calif. (1990)].

“Vectors”, as used herein, encompass plasmids, viruses, bacteriophage, any DNA fragment capable of integration, and other vehicles, which enable the integration of such DNA fragments into the genome of the host. Expression vectors are typically self-replicating DNA or RNA constructs containing the desired gene or its fragments, and operably linked genetic control elements that are recognized in a suitable host cell and effect expression of the desired genes. These control elements influence the expression of the cloned sequences within a suitable host. Generally, the genetic control elements can include a prokaryotic promoter system or a eukaryotic promoter expression control system. This typically includes a transcriptional promoter, an optional operator to control the onset of transcription, transcription enhancers to elevate the level of RNA expression, a sequence that encodes a suitable ribosome binding site, RNA splice junctions, sequences that terminate transcription and translation and so forth. Expression vectors usually contain an origin of replication that allows the vector to replicate independently of the host cell.

A vector may additionally include appropriate restriction sites, antibiotic resistance or other markers for selection of vector containing cells. Plasmids are the most commonly used form of vector but other forms of vectors which serves an equivalent function and which are, or become, known in the art are suitable for use herein. See, e.g., Pouwels et al. Cloning Vectors: a Laboratory Manual (1985 and supplements), Elsevier, N.Y.; and Rodriquez, et al. (eds.) Vectors: a Survey of Molecular Cloning Vectors and their Uses, Buttersworth, Boston, Mass. (1988), which are incorporated herein by reference.

In yet another embodiment, the expression vectors of the invention may comprise any of the nucleic acid sequences as defined by the invention.

Examples 1 and 2 exemplify construction of MEP-Mel using the mammalian expression vector pcDNA3, for the expression of the recombinant protein in DC cells. The same nucleic acid sequence was also subcloned into pQE12 plasmid, to be used as a prokaryotic expression system for the overexpression, production and purification of the recombinant protein. However, it should be appreciated that any other suitable vector may be used.

The invention further provides for a host cell transformed with any of the expression vectors of the invention. Suitable host cells include prokaryotes, lower eukaryotes, and higher eukaryotes. Prokaryotes, which may be used where high levels of protein expression is required, include Gram-negative and Gram-positive organisms, e.g., E. coli and B. subtilis. Lower eukaryotes include yeast, S. cerevisiae and Pichia, and species of the genus Dictyostelium. Higher eukaryotes include established tissue culture cell lines from animal cells, both of non-mammalian origin, e.g., insect cells and birds, and of mammalian origin, e.g., human and other primate, and of rodent origin.

In a particular embodiment, the expression vector host cell may be a mammalian cell. A specifically preferred mammalian host cell, may be an antigen presenting cell (APC) selected from dendritic cells (DC), activated B cells, and activated macrophages.

Several cell types appear to be capable of serving as APC, including dendritic cells (DC), activated B cells, and activated macrophages. In accordance with the invention the APCs are preferably autologous cells and in some illustrative embodiments the antigen-presenting cell may be a dendritic cell (DC). It is understood that one of skill in the art will recognize that other antigen presenting cells may be useful in the invention, such as B cells activated by lipopolysaccharide, whole spleen cells, peripheral blood macrophages, fibroblasts or non-fractionated peripheral blood mononuclear cells (PBMC). Therefore, the invention is not limited to the exemplary cell types which are specifically mentioned and exemplified herein.

Antigen-presenting cells (APCs) present the antigens in association with MHC molecules. Endogenous antigens are processed by the proteolytic system of the cell and presented by Class I MHC molecules. Usually, MHC Class I molecules will display a 8 to 9 amino acid peptide which will be recognized by CD8+ cytotoxic T cells. Exogenous antigens processed to fragments of 12-20 or more amino acid, are presented in association with MHC Class II molecules and recognized by CD4+ T helper cells. The phenonmenon by which CD4+ and CD8+ T cells can recognize an antigen only when it has been processed and presented on the cell membrane of an APC or Target Cell (respectively) in association with self MHC, is termed MHC restriction.

A third aspect of the invention relates to a recombinant multiepitope polypeptide (MEP) wherein said MEP comprises at least two T cell epitopes (antigenic determinant) which may be identical or different, derived from a tumor-associated antigen (TAA). These epitopes are presented by an antigen presenting cell (APC) in the context of MHC Class I molecules and induce T cell activation. Each of these epitopes may be operably linked to an adjacent epitope by a signal for proteasomal cleavage. It should be noted that these signals may be identical or different.

In a specific embodiment, the MEP of the invention may further comprise adjuvants or carriers. Such addition of particular carrier enables presentation of the MEP of the invention by an APC, in the context of any one of MHC Class I and Class II molecules. In a particular embodiment, such carrier may be the Immunoglobulin Fc fragment or an enterotoxin, preferably the E. Coli heat labile enterotoxin subunit B (LTB).

According to a specifically preferred embodiment, the epitopes comprised within the MEP of the invention are derived from TAAs, which are associated with any one of carcinomas, lymphomas, melanomas and sarcomas.

In yet another specifically preferred embodiment, the signals for proteasomal cleavage comprised within the MEP of the invention, direct intracellular proteasomal cleavage of said MEP into epitope peptides presented by an antigen presenting cell (APC) in the context of MHC Class I molecules. The signals for proteasomal cleavage may be according to preferred embodiment, peptides selected from the group consisting of RKSY, RKSYL, ALL, SSL, AAY, AVHV, RVTIL and AASRY substantially as denoted by any one of the amino acid sequences of SEQ ID NO: 19 to 23 and 41 to 43, respectively.

Proteasomal cleavage may produce small peptide segments (8 to 10 aminoacids) representative of the antigenic epitopes of comprised in the MEPs. Alternative cleavage or partial cleavage, may render larger peptide fragments comprised of 12-20 or more amino acids.

In one specific embodiment, the epitopes comprised within the MEP of the invention are derived from any of the melanoma-associated antigens (MAA) tyrosinase, gp-100, MAGE-3 and MART-1.

Preferably, these epitopes may be selected from the group consisting of peptides 280-288 and 209-217 of gp100, peptide 369-377 of tyrosinase and peptide 27-35 of MART-1, substantially as denoted by the amino acid sequences as denoted by SEQ ID NO: 15 to 18, respectively, or by any functional homologue, variant, equivalent and derivative thereof

Therefore, a recombinant multiepitope polypeptide (MEP) of the invention may be for example, a MEP comprising at least two T cell epitopes, which may be identical or different, derived from melanoma-associated antigens selected from the group consisting of amino acids 280-288 of gp100, amino acids 209-217 of gp100, amino acids 369-377 of tyrosinase and amino acids 27-35 of MART-1, substantially as denoted by the amino acid sequences as denoted by SEQ ID NO: 15 to 18, respectively. Each of said epitopes is linked to an adjacent epitope by a signal for proteasomal cleavage. Such signals, which may be identical or different, may be selected from the group consisting of RKSY, RKSYL, ALL, SSL AAY, substantially as denoted by SEQ ID NO: 19 to 23, respectively.

A particular example of a melanoma-derived multiepitope polypeptide, is the MEP designated MEP-Mel, which has the amino acid sequence as denoted by SEQ ID NO: 2 or SEQ ID NO: 57, or by any functional analogue, variant, equivalent and derivative thereof.

In another specific embodiment, the epitopes comprised within the MEP molecule of the invention may be derived from the breast and ovarian carcinoma associated antigens, Mucin-1 (MUC1) and Lactadherin (BA46).

In a preferred embodiment, such recombinant polypeptide may comprise epitopes selected from the group consisting of peptides D6 (LLLTVLTVV) and A7 (NLTISDVSV) of MUC1, and peptides BA46-6 (NLFETPVEA) and BA46-7 (GLQHWVPEL) of Lactadherin, substantially as denoted by the amino acid sequences as denoted by SEQ ID NO: 25 to 28, respectively, or by any functional homologue, variant, equivalent and derivative thereof.

Accordingly, the invention provides for a recombinant multiepitope polypeptide (MEP) comprising at least two T cell epitopes derived from epithelial carcinoma-associated antigens. These epitopes may be identical or different and may be selected from the group consisting of peptides D6 (LLLTVLTVV) and A7 (NLTISDVSV) of MUC1, peptides BA46-6 (NLFETPVEA) and BA46-7 (GLQHWVPEL) of Lactadherin, substantially as denoted by the amino acid sequences of SEQ ID NO: 25 to 28, respectively. Each of said epitopes is linked to an adjacent epitope by a signal for proteasomal cleavage that may be identical or different. Such signal may selected from the group consisting of RKSYL, AAY, AVHV, RVTIL and AASRY substantially as denoted by SEQ ID NO: 20, 23, 41 to 43, respectively.

A particular example for such recombinant polypeptide, is the epithelial MEP designated MEP-Epi having the amino acid sequence as denoted by SEQ ID NO: 40, or by any functional analogue, variant, equivalent and derivative thereof.

It should be noted that any of the recombinant polypeptides of the invention, may be produced synthetically or preferably, may be produced by any of the host cells as defined by the invention.

In a further aspect, the invention relates to a composition for inducing an immune response directed against malignancy in a mammalian subject. The compositions of the invention may comprise as an active ingredient any one of the multiepitope polypeptide (MEP) of the invention, any one of the LTB-MEP fusion proteins, a recombinant nucleic acid sequence encoding any said polypeptides, an expression vector encoding said MEP and LTB-MEP fusion proteins, and a host cell transfected with any of said vectors as defined by the invention.

The invention further provides a pharmaceutical composition for the treatment of a malignant disorder in a mammalian subject. The pharmaceutical composition of the invention may comprise as an active ingredient the multiepitope polypeptide (MEP) of the invention, or the LTB-MEP fusion proteins, a recombinant nucleic acid sequence encoding any of said polypeptides, an expression vector encoding said MEP and LTB-MEP fusion proteins, and a host cell transfected with any of said vectors as defined by the invention.

The compositions of the invention may optionally further comprise pharmaceutically acceptable carrier, diluent, excipient and/or additive.

The active agents of the present invention may be administered directly to the subject to be treated or it may be desirable to conjugate them to carrier proteins or adjuvants prior to their administration. Therapeutic formulations may be administered in any conventional dosage formulation. Formulations typically comprise at least one active ingredient, as defined above, together with one or more acceptable carriers thereof.

Composition dosages may be any that induce an immune response. It is understood by the skilled artisan that the preferred dosage would be individualized to the patient following good laboratory practice and standard medical practice. The preferred single dosage comprises an amount of 1-20 mg of the active ingredient for injections and topical treatments.

Each carrier should be both pharmaceutically and physiologically acceptable in the sense of being compatible with the other ingredients and not injurious to the patient. While formulations include those suitable for topical, oral, rectal, nasal, preferred formulations are intended for parenteral administration, including intramuscular, intravenous, intradermal and specifically subcutaneous administration. The formulations may conveniently be presented in unit dosage form and may be prepared by any methods known in the art of pharmacy.

Thus, in a specific embodiment, the compositions of the invention may further comprise an IgG Fc fragment and enterotoxin, preferably LTB, mixed with said active ingredient.

Such carriers could be an integral part of the polypeptide or chemically linked thereto. Chemically linked carriers, may be associated by a chemical linker to the optional recombinant MEP of the invention. Cross-linking proteins is well known in the art, see e.g., Chemistry of Protein Conjugation and Cross-linking, Shan S. Wong, CRC Press, 1991. Proteins may be cross-linked by their functional groups. Usually, the SH or NH2 groups of proteins are used for that purpose. Chemical groups that react with SH groups include e.g., dithio groups, including pyridyldithio groups, haloacetamido groups, including iodoacetamido groups, maleimido groups, including alkylmaleimido groups, and the like groups known to the skilled person. Amino groups may be coupled using optionally sulfonated N-hydroxysuccinimide ester groups, imidoester groups, including methyl pimelimidate and methyl suberimidate groups, or carbodiimide groups. Also free carboxyl groups of a protein may be used for cross-linking, e.g. using an amino group such as an alkylamino group, and providing a dehydrating agent in the reaction.

The cross-linker used for proper association of the MEP with a desired carrier, may be homobifunctional or heterobifunctional. Examples for homobifunctional cross-linkers include disuccinimidyl suberate (DSS), disuccinimidyl glutarate (DSG) and dimethyl suberimidate (DMS). Examples for heterobifunctional cross-linkers include m-maleimideobenzoyl-N-hydroxysuccinimide ester (MBS) and N-gamma-maleimidobutyryloxy-succinimide ester (GMBS). In yet another embodiment of the invention, a cross-linker is capable of reacting unspecifically with proteins, for instance by photoactivation. Examples for photoreactive groups are e.g., the azidobenzoyl, azido-nitrobenzoyl, azido-hydroxybenzoyl or azido-coumarin groups. Examples of photoreactive cross-linkers include p-nitrophenyl-2-diazo-3, 3, 3-trifluoropropionate (PNP-DTP) and azidobenzoyl hydrazide.

A carbohydrate-reactive cross-linker may be also used. Carbohydrate reactive groups include e.g., the aldehyde group, the glyoxal group, or the sulfone group. Cross-linkers reactive with carbohydrates include e.g., the above azidobenzoyl hydrazide, 4-[m-maleimidomethyl]-cyclohexane-1-carboxyl-hydrazide (M2C2H), or 4-(4-N-maleimidophenyl)-butyric acid hydrazide (MPBH). If any of the MEP of the invention or the carrier does not comprise cysteine residues, a photoreactive cross-linker or an amino-reactive cross-linker may be used, such as the activated N-hydroxy succinimide derivative of the above M2C2H or MPBH, e.g., 4-(4-(succinimido-N-oxo)-phenyl)-butyric acid hydrazide. Alternatively, when it is desired to use the above carbohydrate and sulfhydryl-reactive cross-linkers, a second cross-linker may be used, which may be linked to the sulfhydryl-reactive moiety of the first cross-linker. The protein may then be coupled via the second functionality of the second cross-linker, which advantageously is a group reactive with amino groups, such as an activated N-hydroxy succinimide ester group.

The above noted cross-linkers are commercially available, e.g., from PIERCE, as listed at p. O-90 to O-104 of the 1994 Life Sciences Product Catalog and Handbook of PIERCE, Rockford, Ill. 61105 USA, or from other suppliers in the field of organic chemistry, such as e.g., Sigma, St. Louis, USA.

U.S. Pat. No. 5,399,501 describes the conjugation of immunologically active proteins, e.g. antibodies, to a solid phase via a rather elaborate set of three distinct molecules: first, a cross-linker which binds to amino, carboxyl or thiol groups on the surface of the solid phase and provides a group capable of reacting with thiols (e.g. maleimide); second, a cross-linker that binds to NH2 groups of the protein to be conjugated and also provides a group capable of reacting with thiols (e.g. maleimide), and third, a dithiol reagent capable of joining the solid-phase bound thiol-reactive group with the protein-bound thiol-reactive group. This set of cross-linkers may also be used in the present invention, for the purpose of cross-linking of the MEP and a desired carrier such as Ig Fc fragment or enterotoxin, preferably LTB, or associating both elements to a connecting component (such as a solid support, beads, etc.).

Association of the MEP and the carrier may be carried out for example using a peptide linker. The peptide linker is a peptide of suitable amino acid sequence, which is expected not to interfere with the secondary and tertiary structure of both components. The linker peptide may be connected to the MEP by a cross-linker, as described above for linking proteins. The necessary functional groups for cross-linking may be provided in the linker peptide by the choice of amino acids. For instance, lysine or arginine is chosen when it is desired to use amino groups for cross-linking. Cysteine residues are chosen when it is desired to use sulfhydryl groups for cross-linking. Glutamic acid or aspartic acid may be chosen when it is desired to use carboxylic acid groups for cross-linking. Groups that are not desired to be reacted may be protected by a suitable protection group as known in the art for amino, carboxyl, or sulfhiydryl groups.

The linker may preferably comprise between 10 and 150 amino acids in length. Further preferably, the linker comprises small, uncharged amino acids, such as glycine, alanine, valine, serine, or threonine.

Alternatively, both MEP and the carrier may be linked via a peptide, by using recombinant DNA technology. This possibility is detailed in Examples 5 and 6. A recombinant nucleic acid sequence encoding a fusion protein is composed of a multiepitope polypeptide (MEP) which sequence comprises at least two segments, wherein each of said segments, which may be identical or different, encodes a T cell epitope derived from a tumor associated antigen (TAA), and wherein each of said segments is operably linked to an adjacent segment by a spacer element, wherein each of said spacer elements, which may be identical or different, encodes a signal for proteasomal cleavage, operably linked via a suitable linking element to a nucleic acid sequence encoding LTB (SEQ ID NO: 52).

The LTB-MEP fusion proteins described herein include an LTB-melanoma-derived multiepitope fusion protein (LTB-MEP-Mel), LTB-breast cancer derived multiepitope fusion protein (LTB-MEP-Epi), or any functional analogue, variant, equivalent and derivative thereof.

The recombinant nucleic acid sequence encoding the LTB-MEP-Mel fusion protein comprises a MEP sequence composed of at least two segments, which may be identical or different, wherein each of said segments encodes T cell epitopes derived from melanoma associated antigens selected from the group consisting of amino acids 280-288 of gp100, amino acids 209-217 of gp100, amino acids 369-377 of tyrosinase and amino acids 27-35 of MART-1, substantially as denoted by the amino acid sequences as denoted by SEQ ID NO: 15 to 18, respectively, and wherein each of said segments is operably linked to an adjacent segment by a spacer element, wherein each of said spacer elements, which may be identical or different, encodes a signal for proteasomal cleavage selected from the group consisting of RKSY, RKSYL, ALL, SSL and AAY, substantially as denoted by SEQ ID NO: 19 to 23, respectively, operably linked by a synthetic linker to the LTB encoding sequence (SEQ ID NO: 52).

The recombinant nucleic acid sequence encoding the LTB-MEP-Mel fusion protein comprises a MEP sequence composed of at least two segments, which may be identical or different, wherein each of said segments encodes T cell epitopes derived from breast and ovarian carcinoma-associated antigens selected from the group consisting of peptides D6 (LLLTVLTVV) and A7 (NLTISDVSV) of MUC1, peptides BA46-6 (NLFETPVEA) and BA46-7 (GLQHWVPEL) of Lactadherin, substantially as denoted by the amino acid sequences as denoted by SEQ ID NO: 25 to 28, respectively, and wherein each of said segments is operably linked to an adjacent segment by a spacer element, wherein each of said spacer elements, which may be identical or different, encodes a signal for proteasomal cleavage selected from the group consisting of: RKSYL, AAY, AVHV, RVTIL and AASRY, substantially as denoted by any one of SEQ ID NO: 20, 23 and 41 to 43, respectively, operably linked by a synthetic linker to the LTB encoding sequence.

Such recombinant protein, termed “LTB-MEP fusion protein” in the invention, may be produced using an expression system consisting of a host cell transfected with an expression vector encoding said LTB-MEP fusion protein.

The composition of the invention is particularly directed at inducing immune response against carcinomas, lymphomas, melanomas and sarcomas. For example, prostate, ovary, kidney, lung, brain, breast, colon, bone, skin, testes and uterus cancer may be treated, and most preferably, melanoma.

The MEPs and fusion proteins of the invention act as pro-drugs, releasing the active agents after metabolism and their post-administration proteasomal cleavage. Once antigenic products of this cleavage are formed, they are presented on APCs in the context of MHC Class I and Class II molecules, and thereby elicit the desired immune response.

The term melanoma includes, but is not limited to, melanoma, metastatic melanoma, melanoma derived from either melanocytes or melanocyte-related nevus cells, melanocarcinoma, melanoepithelioma, melanosarcoma, melanoma in situ, superficial spreading melanoma, nodular melanoma, lentigo maligna melanoma, acral lentiginoous melanoma, invasive melanoma or familial atypical mole and melanoma (FAM-M) syndrome. Such melanomas may be caused by chromosomal abnormalities, degenerative growth and developmental disorders, mitogenic agents, ultraviolet radiation (UV), viral infections, inappropriate tissue gene expression, alterations in gene expression, or carcinogenic agents. The aforementioned melanomas can be treated by the method and the composition described in the present invention.

As used herein to describe the present invention, “cancer”, “tumor” “malignant disorder” and “malignancy” all relate equivalently to a hyperplasia of a tissue or organ. If the tissue is a part of the lymphatic or immune systems, malignant cells may include non-solid tumors of circulating cells. Malignancies of other tissues or organs may produce solid tumors.

In a particular embodiment, the compositions of the invention may be used where the malignant disorder is melanoma.

The compositions of the invention are particularly intended for the induction of immune response in a mammalian subject, preferably, in humans, but other mammals including, but not limited to, monkeys, equines, cattle, canines, felines, mice, rats, pigs, horses, sheep and goats may be treated.

In yet another preferred embodiment, the APC comprised as an active ingredient, within the composition of the invention, may be autologous dendritic cells (DC). It should be noted that these cells may be transfected with the nucleic acid sequence or the expression vectors of the invention, or alternatively, may be loaded with the MEP or LTB-MEP fusion protein of the invention.

The compositions of the invention can be administered in a variety of ways. By way of non-limiting example, the composition may be delivered transdermally, intravenously, or into a body cavity adjacent to the location of a solid tumor, such as the intraperitoneal cavity, or injected directly into or adjacent to a solid tumor. Intravenous administration, for example, is advantageous in the treatment of leukemias, lymphomas, and comparable malignancies of the lymphatic system.

For all administrations, conventional depot forms are suitably used. Such forms include for example, microcapsules, nano-capsules, liposomes, inhalation forms, nose sprays and sustained-release preparations.

As a preferred route the composition of the present invention may be administered via subcutaneous or intradermal injections in proximity to the tumor, via intralymphatic or intravenous injection.

The pharmaceutical forms suitable for injection use include sterile aqueous solutions or dispersions and sterile powders for the extemporaneous preparation of sterile injectable solutions or dispersions. In all cases the form must be sterile and must be fluid to the extent that easy syringeability exists. It must be stable under the conditions of manufacture and storage and must be preserved against the contaminating action of microorganisms, such as bacteria and fungi. The carrier can be solvent or dispersion medium containing, for example, water, ethanol, polyol (for example, glycerol, propylene glycol, and liquid polyethylen glyol, and the like), suitable mixtures thereof, and vegetable oils. The proper fluidity can be maintained, for example, by the use of a coating, such as lecithin, by the maintenance of the required particle size in the case of dispersion and by the use of surfactants.

The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above.

In the case of sterile powders for the preparation of the sterile injectable solutions, the preferred method of preparation are vacuum-drying and freeze drying techniques which yield a powder of the active ingredient plus any additional desired ingredient from a previously sterile-filtered solution thereof.

In case of topical application, the composition may be supplied in the form of ointment, cream, spray, patches or sustained-release patches. Other suitable administration vehicles may include osmotic pumps, microcapsules, nano-capsules, liposomes, inhalation forms, nose sprays.

As used herein “pharmaceutically acceptable carrier” includes any and all solvents, dispersion media, coatings, antibacterial and antifungal agents and the like. The use of such media and agents for pharmaceutical active substances is well known in the art. Except as any conventional media or agent is incompatible with the active ingredient, its use in the therapeutic composition is contemplated.

Supplementary active ingredients can also be incorporated into the compositions.

Although it is not envisioned as a preferred route, the composition of the invention or its active ingredients, the MEP, transfected host cells or MEP pulsed DC, may also be orally administered, for example, with an inert diluent or with an assimilable carrier, or enclosed in hard or soft shell gelatin capsule, or compressed into tablets, or incorporated directly with the food of the diet.

Another aspect of the invention relates to a method for conferring immunity against a malignancy in a mammalian subject, comprising the steps of administering to a subject in need, the multiepitope polypeptide (MEP) of the invention, a recombinant nucleic acid sequence encoding said MEP or LTB-MEP, an expression vector encoding said MEP, a host cell transfected with said vector, an autologous APC loaded with said MEP or LTB-MEP, or a composition comprising the same as defined by the invention, in an amount sufficient to induce in said subject an immune response against said malignancy.

The invention also relates to a transdermal drug delivery system for the treatment of a malignant disorder. This system comprises any one of the LTB-MEP fusion protein as described above as the active ingredient, optionally further comprising pharmaceutically acceptable carrier, diluent, excipient, adjuvant and additive, administered in the form of ointment, cream, spray, patches, sustained-release patches, osmotic pumps or any other suitable vehicle. This transdermal drug delivery system is capable of inducing a systemic antigen specific immune response in a mammalian subject in need.

The invention further provides for a method for the treatment of a malignant disorder in a mammalian subject in need. This method comprises the step of administering to said subject the multiepitope polypeptide (MEP) of the invention, a recombinant nucleic acid sequence encoding said MEP, an expression vector encoding said MEP, a host cell transfected with said vector, an autologous APC loaded with said MEP or a composition comprising the same as defined by the invention, in an amount sufficient to induce in said subject an immune response against said

Another method for the treatment of a malignant disorder in a mammalian subject in need is provided in the invention. Such method comprises the step of applying to said subject a transdermal drug delivery system as defined above, in a amount sufficient to induce in said subject a systemic immune response against said malignancy. Said immune response results in the production of antibodies, helper and cytotoxic T lymphocytes, specific for the different antigens associated with said malignancy, comprised in said LTB-MEP fusion protein.

As used herein, “effective amount” means an amount necessary to achieve a selected result. The “effective treatment amount” is determined by the severity of the disease in conjunction with the therapeutic objectives, the route of administration and the patient's general condition (age, sex, weight and other considerations known to the attending physician). For example, an effective amount of the composition of the invention useful for the treatment of said pathology will be an amount sufficient to induce effective immune response necessary to achieve a selected result. For example, an effective amount of the composition of the invention will be conferring immunity against the treated malignant disorder.

The method of the invention may be applicable for malignancies such as carcinomas, lymphomas, melanomas and sarcomas.

In a particular embodiment, the methods of the invention are applicable where the malignancy or malignant disorder is melanoma.

“Treatment” refers to therapeutic treatment. Those in need of treatment are mammal subjects suffering from a tumorogenic disease.

In a preferred embodiment, the method of the invention is intended for treating a mammalian subject, preferably, a human. Therefore, by “patient” or “subject in need” is meant any mammal for which gene therapy is desired, including human bovine, equine, canine, and feline subjects, preferably, human patient.

According to a particular embodiment, where host cell transfected with the vectors of the invention are administered to a subject in need according to the method of the invention, such cells may be preferably autologous APCs. Most preferably, autologous dendritic cell (DC).

It is further contemplated that in practising the invention one may wish to alter the DCs by ex vivo manipulation. In such ex vivo protocols, the biological sample, particularly a blood sample, may be drawn from the body of the human subject by methods known to the skilled artisan in the fields of oncology and surgery, and include sampling blood in well-known ways.

Briefly, blood is drawn from the patient by cytopheresis, a procedure by which a large number of white cells are obtained, while other blood components are being simultaneously transferred back to the patient. The composition of the invention may be prepared from these cells and frozen in small aliquots.

It should be appreciated that the immune response initiated by any of the methods of the invention results in a cellular (involves T cells) and/or humoral (B cells mediated) response. This response may induce the production of both helper and cytotoxic T lymphocytes, as well as antibodies specific for different antigens associated with said malignancy which are comprised in the MEP or LTB-MEP fusion proteins of the invention. Said immune response should be systemic, although the treatment will be provided in the form of local application.

For the in vivo treatment in accordance with the invention, the compositions of the invention can be administered in a variety of ways. By way of non limiting example, the MEP, expression vector, host cells or compositions of the invention may be delivered intravenously, or into a body cavity adjacent to the location of a solid tumor, such as the intraperitoneal cavity, or injected directly into or adjacent to a solid tumor. Intravenous administration, for example, is advantageous in the treatment of leukemias, lymphomas, and comparable malignancies of the lymphatic system.

Still further, the invention relates to the use of a multiepitope polypeptide (MEP) capable of being presented by an antigen presenting cell (APC) in the context of any one of MHC Class I and Class II, in the preparation of a pharmaceutical composition defined by the invention and a transdermal drug delivery system, for the treatment of a malignant disorder.

The invention further provides a new approach for the treatment against tumors. Local topical application of an LTB-MEP fusion protein induces a systemic immunological response. This simple, not invasive external way of administering a treatment, probably results in minor secondary effects which may avoid patient discomfort, well known in the alternative radio and chemotherapy conventional treatments.

As used in the specifications and the appended claims and in accordance with long-standing patent Law practice, the singular forms “a” “an” and “the” generally mean “at least one”, “one or more”, and other plural references unless the context clearly dictates otherwise. Thus, for example “a cell”, “a peptide” and “an adjuvant” include mixture of cells, one or more peptides and a plurality of adjuvants of the type described.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The contents of all publications quoted to herein are fully incorporated by reference.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

Experimental Procedures

Vectors, Enzymes and kits

pcDNA3—purchesed from Qiagene CAT. NO. V79020.

pQE30—purchesed from Qiagene (CAT. NO. 32915.)

DNA Mini preparation kit—purchesed from Promega (CAT. NO.732-6100)

Rapid Gel Extraction kit—for isolation and extraction of PCR products from agarose gel, purchesed from CibcoBRL CAT. NO.11456-019.

Restriction Enzymes: BamHI, Hind III, EcoRI and KpnI, were purchased from New England Biolab.

FBI-HS Polymerase purchased from Fish biotc Australia was used for PCR.

nucleotides for PCR reaction were purchased from PROMEGA.

Sequencing—performed by MBC Rehovot, Israel.

Construction of the MEP-Mel Nucleic Acid Fragment (495 bp)

The desired nucleic acid fragment was constructed using two PCR steps. First, different PCR reactions were performed using the 12 primers (as denoted by SEQ ID NO:3-14), primers 1-4 produced a 157 bp product, primers 5-7 produced a 175 bp product and primers 8-12 produced a 200 bp product. Finally, the resulting PCR product was amplified using the most external 5′ and 3′ primers.

Ligation and Transformtion of MEP-Mel into E. coli JM109

The purified MEP-Mel digested fragment and the pcDNA3 vector were ligated and transformed into E. coli JM109. The transformed bacteria were selected on Ampicillin-LB plates. Bacteria transformed with the vector were used as control.

Colonies carrying the desired PCR product were identified by PCR amplification, using the primers of both ends of the insert and by digestion of plasmid by EcoRI and KpnI to release the insert. Positive colonies were further subjected to sequence analysis.

Production of Recombinant Protein

Bacteria carrying the MEP-Mel-containing plasmid with the correct sequence were grown on LB medium supplemented with 100 μg/ml of ampicilin to OD 0.5-0.7. Production of recombinant protein was induced by addition of 1 mM IPTG (Isopropyl β-D-thiogalactoside) to the medium and growing the bacteria for 3 hours at 37° C. The cells were centrifuged, and the pellet was dissolved in disruption buffer (50 m M Tris-HCl, 50 m M NaCl, 1 m M EDTA) and lysozyme (10 mg/ml) and incubated for 30 min. at 4° C. with gentle shaking. This was followed by addition of 10 mg/ml DNAse, 20 mg/ml deoxycholine and 1M MgCl₂ and incubation at room temperature for 30 min. The disrupted bacteria were centrifuged (30 min, 4° C., 17000 g). The pellet contained the MEP-Mel polypeptide in inclusion bodies (IB). IB were partially cleaned by shaking in wash buffer (50 m M Tris-HCl, 50 m M NaCl, 1 m M EDTA, 2M urea) and centrifugation (30 min, 4° C., 17000 g). This step was repeated, and then the pellet was dissolved in solubilization buffer (50 m M Tris-HCl, 50 m M NaCl, 1 m M EDTA, 4M/6M/8M urea), shaken for 4 hours and centrifuged (30 min, 4° C., 17000 g).

SDS-Polyacrylamide Gel Electrophoresis (SDS-PAGE) and Western Blotting

Loading buffer (3%, w/v SDS and 5%, v/v mercaptoethanol) was added to each sample, which was then boiled, and electrophoresed in 15% polyacrylamide slab gels, using a discontinuous SDS gel system (Bio Rad, Hercules, Calif.). In most cases, two slab gels were electrophoresed simultaneously. One was stained with Coomassie Brilliant Blue R, and the proteins from the second gel were electrotransferred onto a nitrocellulose filter (Hybond C, Amersham International) using a semi-dry system (Bio Rad) for Western blotting. Following blocking with milk buffer, the membrane was incubated for 1 h at 37° C. with mouse anti polyhistidine (Sigma) diluted 1:2000 in milk buffer. Filters were washed twice in PBS and incubated with goat anti-mouse IgG-peroxidase conjugate (Sigma) diluted 1:1000, followed by incubation with substrate solution 3,3′-diaminobenzidine (Sigma).

Isolation of DC (Human Dendritic Cells)

Dendritic cells were isolated from cytopheresis MNCs of melanoma patients with the HLA haplotypes HIA-A*0201 and HLA-DRβ1*0401. DC were generated from plastic adherent PBMCs cultured in RPMI with 10% human AB-serum, L-glutamine, penicillin-streptomycin, supplemented with GM-CSF 1000 IU/ml and IL-4 1000 IU/ml. By day 6, immature DC were harvested for electroporation.

Transfection Method

The following DNA plasmids were used: pc DNA-MEP, pcDNA3-gp100, pcDNA3-MART-1, pcDNA3-tyrosinase and pcDNA3-GFP (green fluorescent protein).

DNA plasmids electroporation was performed in the following way:

2.5-3×10⁶ DC were re-suspended in 100 μl nucleofector solution and added to 5 μg of DNA plasmid in 2-mm electrode gap cuvettes. Transfected DC were immediately plated with CM containing GM/IL4 or let mature using TNFα 100 ng/ml and PGE2 1000 ng/ml. GFP-transfected DCs were collected 16-24 hours later for FACS analysis.

Gp100 RNA was in vitro transcribed from a linearized pGEM4Z-64A DNA plasmid. Six days cultured immature DC were re-suspended in Opti-MEM at 2.5×10⁷/ml. One hundred microliter (100 μl) cell suspension was mixed with 5 μg RNA and electroporated in 2 mm cuvette using BTX electroporator. After electroporation, DC were incubated in conditioned medium supplemented with GM-CSF and IL-4. FACS analysis was performed 24 hours later.

pcDNA-MEP and pcDNA-gp100 were co-transfected to detect Class II antigen presentation as an internal control.

DC Pulsing with Peptides

DC were pulsed with 1 μg/ml of the relevant peptides at 1×10⁶ cells/ml. Stimulants were added after 1 hour incubation without washing the peptide.

T Cell Clones and TILs

Patients T cell clones from PBMCs or tumor infiltrating lymphocytes (TILs) were grown. The clones that were used in these experiments are:

CK3H6 which recognizes the A2 restricted gp100 epitope 209-217.

HT 2D9 which reacts with A2 restricted gp100 epitope 280-288.

BR-B8 which recognizes the DR4-restricted gp100 epitope 44-59.

TIL 1940 which recognizes the A2 restricted MART-1 epitope 27-35.

TIL 1383 which recognizes the A2 restricted tyrosinase epitope 369-377.

Recognition Assay for Peptide Presentation by DC

T cell clones were co-incubated with DC in flat 96-well plate for 24 hours. DC were plated immediately after their transfection or pulsing in 6 separated plates, in the presence of GM CSF and IL-4. 1×10⁵ T cells were added to 1×10⁵ pulsed or transfected DC. In the longitudinal study, T cell clones were added to the transfected/pulsed DC at the following time points: T0=immediately, +12=after 12 hours, 24, 36, 48 and 72 hours. Supernatants were collected after overnight incubation. Interferon gamma release assay was performed to evaluate T cell stimulation using the ELISA method, results are presented in pg/ml.

Proteasome Inhibition

Lactacystin was added to DC at concentrations of 25 μM and 50 μM, 4 hours post transfection. DC were incubated with lactacystin for 70 minutes and then washed twice.

Responding T cells were added after washing the lactacystin and co-incubated. Supernatents were collected after 20 hours.

Control DC were transfected and incubated with responder T cell clones within the same time frame, without lactacystin.

Production of LTB-MEP Fusion Protein Construct

The BamHI-HindIII MEP and KpnI-HindII LTB encoding sequences fragments were purified from cloned plasmids. These fragments were amplified using a downstream (3′) primer for LTB and a upstream (5′) primer for MEP with protruding ends which included restriction enzymes recognition sequences. The restriction enzyme digested PCR products (SEQ ID NO: 44-45 and 48-49) were linked together by a linker sequence DNA fragment containing protruding ends complementary to the digested PCR products (SEQ ID NO: 46-47). The resulting LTB-linker-MEP ligated segment is denoted by SEQ ID NO:50-51.

LTB-MEP Fusion Protein Functional Assay

LTB-MEP fusion protein expressed by the recombinant pQE30-LTB-MEP construct was tested for its ability to bind GM1 ganglioside and anti-MEP antibodies. LTB-MEP fusion protein was incubated in ELISA plates coated with GM1 ganglioside (which binds to native LTB) with chicken anti-MEP antibodies.

Only LTB-MEP molecules with functional LTB and MEP units can be detected by this assay. Boiled LTB-MEP protein losses its capability to bind to GM1 ganglioside and therefore could not be detected.

Anti-LTB-MEP Antibody Detection Assay

Anti-LTB-MEP antibody production was evaluated using a sandwich ELISA assay.

C57Bl mice were topically treated with native LTB-MEP, boiled LTB-MEP or PBS. The treatment was applied to mice ears, three times every two weeks. Antibodies titer was measured in mice serum (collected from mice tail bleeding) previously to each substance application. Mice serum was assayed in a plate coated with MEP attached to it through chicken anti-gp100 peptide 280-288 antibodies. The mice antibody binding extent to the attached MEP, was examined with a HRP conjugated goat anti-mouse antibody.

### Example 1

### Preparation of the MEP-Mel Multi Epitope Polypeptide

Construction of the pCDNA3-MEP-Mel Eukaryotic Expression Vector

In order to create a nucleic acid sequence encoding the desired melanoma multiepitope polypeptide, twelve primers were designed and purchased (Sigma). Each primer had an average size of 65 bp, and encoded one or two out of four known melanoma epitope peptides derived from gp100, Tyrosinase and MART-1 (the epitope peptides used herein are also denoted by SEQ ID NO: 15-18) as well as at least one out of five different spacers that served as signals for proteosomal cleavage (also denoted by SEQ ID NO: 19-23). Each primer has an overlapping region with the neighboring primer in the direction of the synthesis, such that each primer serves as a primer and also as a template for the overlapping appropriate primer. Following a series of PCR reactions using these primers (denoted by SEQ ID NO: 3-14), a synthetic 495bp long nucleic acid sequence (also denoted by SEQ ID NO:1) encoding the multiepitope MEP-Mel polypeptide (denoted by SEQ ID NO: 2 or SEQ ID NO: 57), was constructed. It should be further noted that restriction enzyme sites were incorporated at the 5′ and 3′ ends of this nucleic acid sequence. These sites were used for subcloning into the appropriate vector.

The inventors next subcloned the MEP-Mel isolated PCR product into the pcDNA3 expression vector using the EcoRI and KpnI restriction enzymes sites. The resulting recombinant plasmids were transformed into E. coli JM109 and colonies carrying the desired plasmid were isolated and sequenced. This plasmid was used for transfecting DC obtained from patients.

Construction of the pQE30-MEP-Mel Plasmid and Expression MEP-Mel Recombinant Protein

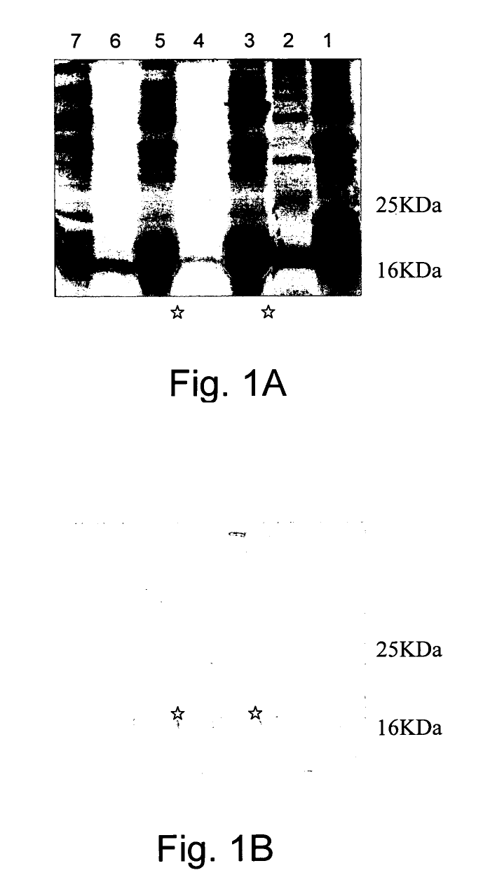

MEP-Mel insert was isolated from pcDNA3 plasmid using the BamHI and HindIII restriction enzymes. The insert was ligated into pQE30 plasmid which allows high yield expression of the desired protein and also contains the His6 tag which allows direct labeling of the desired protein product. A positive colony of JM109 containing pQE30 with MEP-Mel was isolated, grown overnight and MEP-Mel protein was expressed following induction by 0.1M IPTG for 3 hours. Bacteria were disrupted by lysozyme, centrifuged and supernatant and pellet were collected separately. Both fractions were tested by SDS-PAGE stained by Coomassie blue (FIG. 1A) and immunoblot using anti-histidine antibodies (FIG. 1B). MEP-Mel protein was found in the pellet in the form of inclusion bodies.

### Example 2

### Functional Evaluation of MEP-Mel

In order to evaluate the efficacy of presentation of the multiepitope polypeptide of the invention by APC (antigen presenting cell) and its ability to induce specific T cell proliferation, DC isolated from cytopheresis PBMC of two melanoma patients (donor 1 and donor 2) carrying the HLA-A*0201 allele, were used. The PcDNA3-MEP-Mel expression plasmid was transfected to 6 days cultured immature DC, using electroporation as described in Experimental procedures. DC were islolated from melanomas of two different patients. Transfected DC were immediately plated with medium containing GM-CSF/IL4-4 or induced to mature using TNFα (100 ng/ml) and PGE2 (1000 ng/ml).

Efficiency of epitope presentation and stimulation of specific T cell clone proliferation were evaluated by comparing the specific T cell clones response to the DC transfected with the multiepitope, to DC that were pulsed with specific peptides or DC transfected with full length melanoma associated antigens (gp100 and MART-1), using the recognition assay.

For recognition assay of DC epitope presentation, 1×10⁵ cells from different T cell clones, that were grown from PBMCs of patients, and were restricted to particular melanoma antigen peptides, were co-incubated with 1×10⁵ pulsed or transfected DC, in the presence of GM-CSF and IL-4. T cell clones were added to the transfected/pulsed DC at the following time points: T=0 immediately, T=12, 24, 36, 48, 72 hours. Supernatant was collected after overnight incubation and was subjected to interferon gamma release assay, in order to evaluate the specific T cell activity towards the particular peptide presented by the DC cells.

Table 1 shows appropriate recognition of DC pulsed with four different peptides (peptides 154-162 and 209-217 of gp100, peptide 27-35 of MART-1 and peptide 369-377 derived from Tyrosinase, as also denoted by SEQ ID NO: 24, 16, 18 and 17, respectively) by their specific T cell clones (clone RB-154 specific for the gp100 154-162 peptide (SEQ ID NO: 24), clone CK3H6 specific for the gp100 209-217 peptide (SEQ ID NO: 16), TIL 11940 clone specific for the MART-1 27-35 peptide (SEQ ID NO: 18) and TIL 1383 specific for the Tyrosinase 369-377 peptide (SEQ ID NO: 17)).

As shown in Tables 2 and 3, DC that were isolated from two different individuals and that were electroporated with gp100 DNA and gp100 RNA were unable to stimulate class I-restricted epitope specific T cells. It should be noted that the class II-restricted CD4+T cell clone BRB8 has demonstrated longer and stronger stimulation with the RNA-electroporated DC (not shown) than the DNA electroporated DC. DC electroporated with the MART-1 and tyrosinase DNA were not recognized by TILs 1940 and 1383 respectively. Peptide pulsed DC ceased from stimulating T cells within 24 hours (Table 5). However, MEP-Mel pulsed DC demonstrated stimulatory capacity as far as the last assay, 72 hours post pulsing (Tables 2 to 8).

Thus, these results indicate that the multiepitope peptide of the invention stimulates for more than 72 hours specific T cell clones much more efficiently compared to peptides or the whole antigen. More specifically, gp100 epitope 209-217 and MART-1 epitope 27-35 are strongly presented in a sustained, prolonged fashion that is far superior to peptide pulsing.

The results shown in the following tables represent the amount of Interferon-γ (pg/ml) secreted to the culture medium as an index for T cell stimulation.

TABLE 1
Control Plate for the T Cell Clones

| T Cell | gp 100 | gp 100 | MART-1 | TYR |
|---|---|---|---|---|
| Clone | 154-162 | 209-217 | 27-35 | 369-377 |
| CK3H6 | 7 | >2000 | 1 | 1 |
| TIL 1940 | 6 | 1 | >2000 | 1 |
| TIL 1383 | 6 | 1 | 1 | 69 |
| RB-154 | >2000 | 1 | 1 | 0 |
| BR-B8 | 1 | 1 | 1 | 1 |

TABLE 2
DC from donor 2 at T = 0 hours

| T Cell | | gp 100 | MART-1 | TYR |
|---|---|---|---|---|
| Clone | MEP-mel | DNA | DNA | DNA |
| CK3H6 | >2000 | 63 | 11 | 14 |
| TIL 1940 | >2000 | 10 | 90 | 12 |
| TIL 1383 | 387 | 13 | 9 | 34 |
| RB-154 | 132 | 49 | 11 | 0 |
| BR-B8 | | 676 | | |

TABLE 3
DC from donor 1 at T = 0 hours

| T Cell Clone | MEP-mel | gp 100 DNA | MART DNA | TYR DNA |
|---|---|---|---|---|
| CK3H6 | >2000 | 60 | 14 | 17 |
| TIL 1940 | >2000 | 15 | 51 | 15 |
| TIL 1383 | 111 | 19 | 13 | 28 |
| RB-154 | 66 | 19 | 13 | −1 |
| BR-B8 | | >2000 | | |

TABLE 4
DC from donors 1 & 2 at T = 12 hours

| | T Cell | | | DC + gp100 | DC + MART |
|---|---|---|---|---|---|
| Patient | Clone | MEP-mel | GFP | 209-217 | 27-35 |
| Donor 2 | CK3H6 | >2000 | 71 | >2000 | 10 |
| | TIL 1940 | >2000 | 96 | 12 | 448 |
| | CM | 248 | 31 | 8 | 9 |
| Donor 1 | CK3H6 | >2000 | 54 | >2000 | 11 |
| | TIL 1940 | >2000 | 70 | 10 | 9 |
| | CM | 120 | 36 | 9 | 10 |

TABLE 5
DC from donors 1 & 2 at T = 24 hours

| | T Cell | | | DC + gp100 | DC + MART |
|---|---|---|---|---|---|
| Patient | Clone | MEP-mel | GFP | 209-217 | 27-35 |
| Donor 2 | CK3H6 | >2000 | 57 | 18 | 10 |
| | TIL 1940 | >2000 | 79 | 6 | 254 |
| | CM | 292 | 43 | 6 | 7 |
| Donor 1 | CK3H6 | >2000 | 54 | 31 | 6 |
| | TIL 1940 | >2000 | 39 | 1 | 435 |
| | CM | 94 | 21 | | |

TABLE 6
DC from donors 1 & 2 at T = 36 hours

| | T Cell | | | DC + gp100 | DC + MART |
|---|---|---|---|---|---|
| Patient | Clone | MEP-mel | GFP | 209-217 | 27-35 |
| Donor 2 | CK3H6 | >2000 | 43 | 11 | 8 |
| | TIL 1940 | >2000 | 124 | 7 | 49 |
| | CM | 191 | 90 | 8 | 8 |
| Donor 1 | CK3H6 | >2000 | >2000 | 12 | 10 |
| | TIL 1940 | >2000 | 121 | 13 | 99 |
| | CM | 102 | 38 | | |

TABLE 7
DC from donors 1 & 2 at T = 48 hours

| | T Cell | | | DC + gp100 | DC + MART |
|---|---|---|---|---|---|
| Patient | Clone | MEP-mel | GFP | 209-217 | 27-35 |
| Donor 2 | CK3H6 | >2000 | 27 | 0 | 0 |
| | TIL 1940 | >2000 | 41 | 0 | 0 |
| | CM | 260 | 3 | 0 | 0 |
| Donor 1 | CK3H6 | >2000 | 42 | 0 | 0 |
| | TIL 1940 | >2000 | 53 | 0 | 0 |
| | CM | 33 | 25 | 0 | 0 |

TABLE 8
DC from donors 1 & 2 at T = 72 hours

| | T Cell | | | DC + gp100 | DC + MART |
|---|---|---|---|---|---|
| Patient | Clone | MEP-mel | GFP | 209-217 | 27-35 |
| Donor 2 | CK3H6 | 1328 | 3 | 0 | 0 |
| | TIL 1940 | >2000 | 20 | 0 | 0 |
| | CM | 38 | 3 | 0 | 0 |
| Donor 1 | CK3H6 | 1808 | 0 | 0 | 0 |
| | TIL 1940 | >2000 | 16 | 0 | 0 |
| | CM | 18 | 12 | 0 | 0 |

### Example 3

### Uptake of MEP Protein by DC and Presentation to T Cell Clones

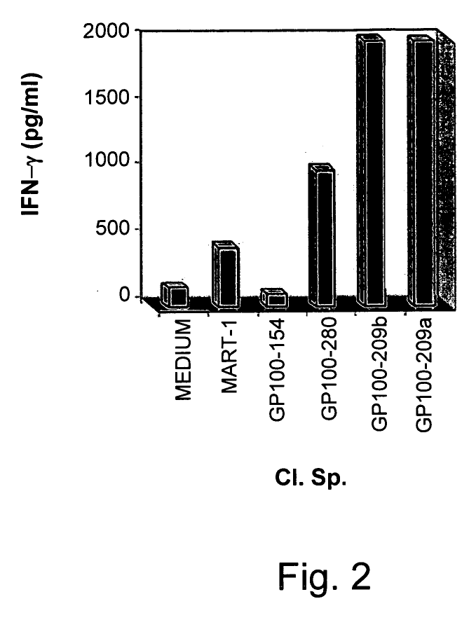

Immature DC were incubated with MEP protein for 4 hours. After DC maturation, dendritic cells were incubated in the presence of T cell clones specific for different gp100 and MART-1 antigens. T cell stimulation was estimated by measuring the interferon gamma (IFN-γ) secretion to the medium. As seen in FIG. 2, two T cell clones specific for gp100 peptide (clone 209a and 209b reactive to epitope 209-217) were highly stimulated. Another T cell clone which recognizes the epitope 280-288 of gp100 protein, was also stimulated. A MART-1 specific T cell clone was also found to be stimulated.

MEP is properly processed in DC and presented to reactive T cell (antigen specific T cell clones).

### Example 4

### Proteasome Cleavage Signals Enables Proper Presentation of the MEP in the Context of Class I Molecules

In order to verify that the proteasome signals inserted as spacers between the different epitopes are recognized by proteasome and that proper presentation of these epitopes (comprised within the multiepitope peptide of the invention) is permitted, the inventors have performed a recognition assay in the presence of Lactacystin, a proteasome inhibitor. As shown in Table 9, proteasomal inhibition decreased the epitope expression of MEP-Mel to background level, indicating that the proteasome sites comprised within the multiepitope peptide of the invention allows appropriate proteasomal cleavage, which enables the proper presentation of these epitopes.

TABLE 9
DC MEP-transfected treated with Lactacystin

| | MEP | | | MEP + gp100 | | | GFP | | CM |
|---|---|---|---|---|---|---|---|---|---|
| T Cell | Lactacystin | | | Lactacystin | | | Lactacystin | | Lactacystin |
| Clone | 0 | 25 μM | 50 μM | 0 | 25 μM | 50 μM | 0 | 25 μM | 0 |
| CK3H6 | 705 | 73 | 36 | 696 | 134 | 116 | 0 | 11 | 0 |
| HT 2D9 | 1642 | 244 | 56 | 1685 | 244 | 171 | 62 | 108 | 62 |
| BR-B8 | 188 | 85 | 16 | 871 | 174 | 328 | 111 | 108 | 145 |
| TIL1941 | 432 | 62 | 45 | 432 | 108 | 139 | 125 | 122 | 91 |
| TIL1383 | 229 | 25 | 0 | 238 | 39 | 96 | 45 | 48 | 96 |
| CM | 5 | 0 | 51 | 0 | 0 | 82 | 0 | 22 | 0 |

### Example 5

### Construction of LTB-MEP-Mel Fusion Protein

A BamHI-HindIII fragment encoding for MEP (SEQ ID NO: 1) was attached to a KpnI-HindIII fragment encoding for LTB (SEQ ID NO: 52) through a polynucleotide linker. The ligated product was cloned into pQE30 vector and transformed into E. Coli (JM109 strain). LTB-MEP fusion protein expressed in bacteria, was purified and further characterized by ELISA assay. Ganglioside GM1 coated plates were incubated at 37° C. in the presence of native or boiled LTB-MEP fusion protein, purified LTB and pQE30 DNA expression vector (without insert) molecules. Binding of the LTB portion of the fusion protein to GM1 ganglioside was observed only in the non-boiled sample (Table 10). LTB molecule is sensitive to temperature and heat treatment destroys its capability to bind to GM1 ganglioside. Since binding detection was performed using chicken anti-MEP antibodies only active LTB-MEP fusion protein could be detected.

TABLE 10
ELISA analysis of LTB-MEP fusion protein

| | OD 450 nm | | |
|---|---|---|---|
| Molecule | LTB-MEP | | |
| Treatment | fusion protein | Purified LTB | pQE30 vector |
| 37° C. | 1.65 | 1.05 | 1.03 |
| Boiled | 1.15 | 0.98 | 0.94 |

LTB-MEP fusion protein can be successfully expressed in vitro and the resulting protein product conserves the biological activities of each of the separated portions of the molecule.

### Example 6

### Transdermal Delivery of LTB-MEP Fusion Protein

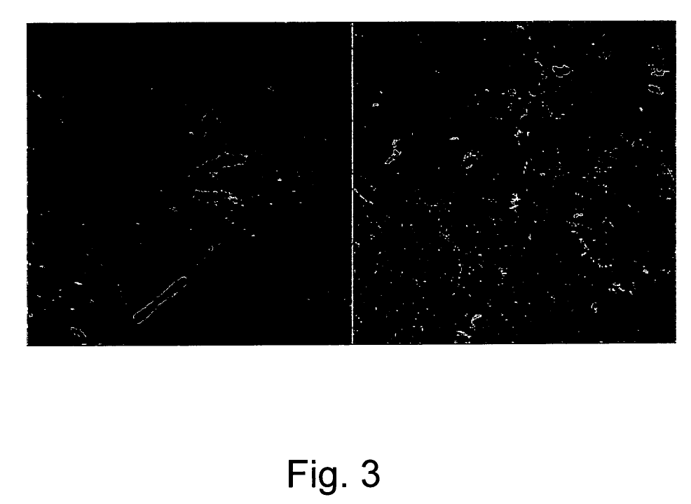

Human foreskin samples were incubated in the presence of FITC-labeled LTB-MEP fusion protein for 30-45 minutes. After separation of the epidermis, fluorescence distribution was examined by confocal microscopy. As seen in FIG. 3, fluorescence was found to be restricted to cells that resemble Langerhans cell morphology. Langerhans cells participate in the cutaneous immune response. They function as antigen presenting cells to T or B lymphocytes by taking up, processing and presenting cutaneous antigens. Langerhans' cells that were able to uptake the LTB-MEP fusion protein, probably will be able to trigger an immunological response against the components of the fusion protein.

### Example 7

### Transdermal Delivery of LTB-MEP Induces Antibody Production

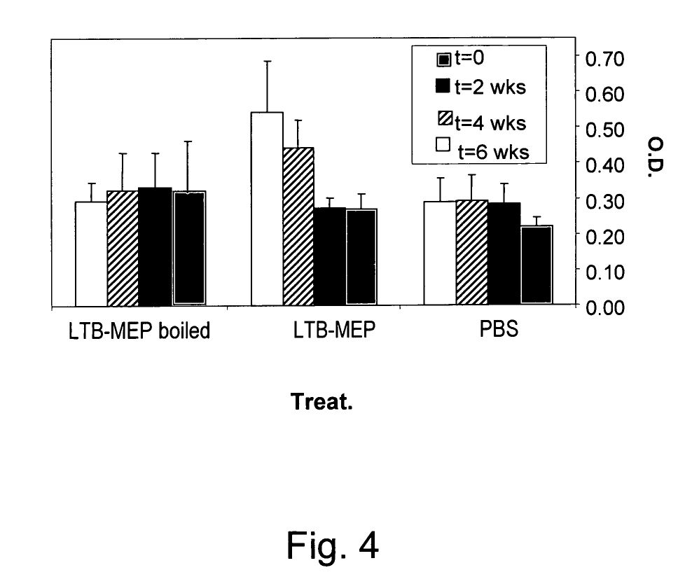

LTB-MEP fusion protein was topically applied on ears of C57B1 mice three times. The mice were tested for anti-LTB-MEP antibody production after each application (two weeks apart). Topical application of LTB-MEP fusion protein induced detectable antibodies production already after the second application (FIG. 4).

These results prove that topical administration of a LTB-MEP fusion protein induces antibody production against epitopes present in the MEP. Therefore, transdermal delivery of LTB-MEP fusion proteins may be applicable as a suitable treatment against tumors with recognized tumor associated antigen (TAA) epitopes.

### Example 8

### Preparation of an Epithelial Multiepitope Peptide MEP-Epi

Construction of the pCDNA3-MEP-Epi Eukaryotic Expression Vector

In order to create a nucleic acid sequence encoding the desired epithelial multiepitope peptide, ten primers were designed and purchased (Sigma). Each primer had an average size of 65 bp, and encoded one or two out of four known breast and ovarian carcinomas epitope peptides derived from MUC 1 and Lactadherin (the epitope peptides used herein are also denoted by the amino acid sequences SEQ ID NO: 25-28) as well as at least one out of five different spacers that served as signals for proteosomal cleavage (also denoted by SEQ ID NO: 20, 23, 41-43). Each primer has an overlapping region with the neighboring primer in the direction of the synthesis, such that each primer serves as a primer and also as a template for the overlapping appropriate primer. Following a series of PCR reactions using these primers (denoted by SEQ ID NO: 29-38), a synthetic nucleic acid sequence (also denoted by SEQ ID NO:39) encoding the multiepitope MEP-Epi polypeptide (denoted by SEQ ID NO: 40), was constructed. It should be further noted that restriction enzyme sites were incorporated at the 5′ and 3′ ends of this nucleic acid sequence, these sites were used for subcloning into the appropriate vector.

The inventors next subcloned the MEP-Epi isolated PCR product into the pcDNA3 expression vector using BamHI and HindIII restriction enzymes sites. The resulting recombinant plasmids were transformed into E. coli JM109 and colonies carrying the desired plasmid were isolated and sequenced. This plasmid was used for transfecting DC obtained from patients.

Construction of the pQE30-MEP-Epi Plasmid and Expression MEP-Epi Recombinant Protein

MEP-Epi insert was digested using the BamHI and HindIII restriction enzymes. The insert was ligated into pQE30 plasmid which allows high yield expression of the desired protein and also contains the His6 tag which allows direct labeling of the desired protein product. A positive colony of JM109 containing pQE30 with MEP-Epi was isolated, grown overnight and MEL-EPI protein was expressed following induction by 0.1M IPTG for 3 hours. Bacteria were disrupted by lysozyme, centrifuged and supernatant and pellet were collected separately.

The invention provides a new approach for the treatment against tumors. The multiple epitope polypeptide was shown to be properly internalized and processed by antigen presenting cells, capable of inducing T cell response and antibody production. It was also shown that topical application of an LTB-MEP fusion protein induces a systemic immunological response. This simple, non invasive external way of administering a treatment may avoid patient discomfort, extensively common in the alternative radio and chemotherapy conventional treatments.

1. A recombinant multiepitope polypeptide (MEP) wherein said MEP comprises at least two T cell epitopes, which may be identical or different, derived from a tumor associated antigen (TAA), and which epitopes are presented by an antigen presenting cell (APC) in the context of MHC Class I molecules and induce T cell activation, wherein each of said epitopes is operably linked to an adjacent epitope by a signal for proteasomal cleavage, which signals may be identical or different.

2. The recombinant polypeptide according to claim 1, further comprising adjuvants or carriers, wherein following proteasomal cleavage, cleavage products of said MEP are presented by an APC in the context of MHC Class I and Class II molecules, preferably, said carrier is any one of enterotoxin and Immunoglobulin Fc fragment, more preferably, said carrier is the E. Coli heat labile enterotoxin (LT), most preferably the B subunit of LT, designated LTB.

3. The recombinant polypeptide according to claim 1, wherein said TAAs are associated with any one of carcinomas, lymphomas, melanomas and sarcomas.

4. The recombinant polypeptide according to claim 3, wherein the signals for proteasomal cleavage, direct intracellular proteasomal excision of said MEP into epitope peptides and/or fragments, which are presented by an antigen presenting cell (APC) in the context of MHC Class I and Class II molecules, preferably, said signals for proteasomal cleavage are peptides selected from the group consisting of RKSY, RKSYL, ALL, SSL, AAY, AVHV, RVTIL and AASRY substantially as denoted by any one of SEQ ID NO: 19 to 23 and 41 to 43.

5. The recombinant polypeptide according to claim 4, wherein said epitopes are derived from any of the melanoma-associated antigens (MAA) tyrosinase, gp-100, MAGE-3 and MART-1, preferably, said epitopes are selected from the group consisting of peptides 280-288 and 209-217 of gp100, peptide 369-377 of tyrosinase and peptide 27-35 of MART-1, substantially as denoted by the amino acid sequences as denoted by SEQ ID NO: 15 to 18, respectively, or any functional homologue, variant, equivalent and derivative thereof.

6. A recombinant multiepitope polypeptide (MEP), wherein said MEP comprises at least two T cell epitopes, which may be identical or different, derived from melanoma-associated antigens selected from the group consisting of amino acids 280-288 of gp100, amino acids 209-217 of gp100, amino acids 369-377 of tyrosinase and amino acids 27-35 of MART-1, substantially as denoted by the amino acid sequences as denoted by SEQ ID NO: 15 to 18, respectively, which epitopes are presented by an antigen presenting cell (APC) in the context of MHC Class I molecules and induce T cell activation, and wherein each of said epitopes is linked to an adjacent epitope by a signal for proteasomal cleavage, which signals may be identical or different, which signal is selected from the group consisting of RKSY, RKSYL, ALL, SSL AAY, substantially as denoted by SEQ ID NO: 19 to 23, respectively.

7. The recombinant polypeptide according to claim 6, wherein said melanoma-associated multiepitope polypeptide, is designated MEP-Mel and has the amino acid sequence as denoted by SEQ ID NO: 2 or SEQ ID NO: 57, or any functional analogue, variant, equivalent and derivative thereof.

8. The recombinant polypeptide according to claim 4, wherein said epitopes are derived from any of the breast and ovarian carcinoma associated antigens Mucin-1 (MUC1) and Lactadherin (BA46), preferably, said epitopes are selected from the group consisting of peptides D6 (LLLTVLTVV) and A7 (NLTISDVSV) of MUC1, and peptides BA46-6 (NLFETPVEA) and BA46-7 (GLQHWVPEL) of Lactadherin, substantially as denoted by the amino acid sequences as denoted by SEQ ID NO: 25 to 28, respectively, or any functional analogue, variant, equivalent and derivative thereof.

9. A recombinant multiepitope polypeptide (MEP) wherein said MEP comprises at least two T cell epitopes derived from epithelial carcinoma-associated antigens, which epitopes may be identical or different and are selected from the group consisting of peptides D6 (LLLTVLTVV) and A7 (NLTISDVSV) of MUC1, peptides BA46-6 (NLFETPVEA) and BA46-7 (GLQHWVPEL) of Lactadherin, substantially as denoted by the amino acid sequences as denoted by SEQ ID NO: 25 to 28, respectively, which epitopes are presented by an antigen presenting cell (APC) in the context of MHC Class I molecules and induce T cell activation, and wherein each of said epitopes is linked to an adjacent epitope by a signal for proteasomal cleavage, which signals may be identical or different, which signal is selected from the group consisting of RKSYL, AAY, AVHV, RVTIL and AASRY substantially as denoted by SEQ ID NO: 20, 23 and 41 to 43, respectively.

10. The recombinant polypeptide according to claim 9, wherein said epithelial carcinoma derived multiepitope polypeptide, is designated MEP-Epi having the amino acid sequence as denoted by SEQ ID NO: 40, or any functional analogue, variant, equivalent and derivative thereof

11. A recombinant polypeptide according to claim 1, wherein said polypeptide is produced by a host cell transfected with an expression vector encoding the polypeptide.

12. A recombinant nucleic acid sequence encoding a fusion protein comprising:
a) a multiepitope polypeptide (MEP) which sequence comprises at least two segments, wherein each of said segments, which may be identical or different, encodes a T cell epitope derived from a tumor associated antigen (TAA), and wherein each of said segments is operably linked to an adjacent segment by a spacer element, wherein each of said spacer elements, which may be identical or different, encodes a signal for proteasomal cleavage, operably linked via a suitable linking element, to
b) a nucleic acid sequence encoding the B subunit of LT, LTB (SEQ ID NO: 52).
wherein following proteasomal cleavage, cleavage products of said fusion protein are presented by an APC in the context of MHC Class I and Class II molecules.

13. The recombinant nucleic acid sequence encoding a fusion protein according to claim 12, wherein said nucleic acid is any one of DNA, RNA and any combination thereof.

14. The recombinant nucleic acid sequence encoding a fusion protein according to claim 13, wherein said TAAs are associated with any one of carcinomas, lymphomas, melanomas and sarcomas.

15. The recombinant nucleic acid sequence encoding a fusion protein according to claim 14, wherein said MEP epitopes are derived from any of the melanoma-associated antigens (MAA) tyrosinase, gp-100, MAGE-3 and MART-1.

16. The recombinant nucleic acid sequence encoding a fusion protein according to claim 15, wherein said MEP sequence comprises at least two segments, which may be identical or different, wherein each of said segments encodes T cell epitopes derived from melanoma associated antigens selected from the group consisting of amino acids 280-288 of gp100, amino acids 209-217 of gp100, amino acids 369-377 of tyrosinase and amino acids 27-35 of MART-1, substantially as denoted by the amino acid sequences as denoted by SEQ ID NO: 15 to 18, respectively, and wherein each of said segments is operably linked to an adjacent segment by a spacer element, wherein each of said spacer elements, which may be identical or different, encodes a signal for proteasomal cleavage selected from the group consisting of RKSY, RKSYL, ALL, SSL and AAY, substantially as denoted by SEQ ID NO: 19 to 23, respectively, preferably, said sequence is denoted by SEQ ID NO: 54 and encodes a LTB-melanoma-derived multiepitope fusion protein, designated LTB-MEP-Mel having the amino acid sequence as denoted by SEQ ID NO: 56, or any functional analogue, variant, equivalent and derivative thereof.

17. The recombinant nucleic acid sequence encoding a fusion protein according to claim 14, wherein said MEP epitopes are derived from any of the breast and ovarian carcinoma associated antigens Mucin-1 (MUC1) and Lactadherin (BA46).

18. The recombinant nucleic acid sequence encoding a fusion protein according to claim 17, wherein said MEP sequence comprises at least two segments, which may be identical or different, wherein each of said segments encodes T cell epitopes derived from breast and ovarian carcinoma-associated antigens selected from the group consisting of peptides D6 (LLLTVLTVV) and A7 (NLTISDVSV) of MUC1, peptides BA46-6 (NLFETPVEA) and BA46-7 (GLQHVWVPEL) of Lactadherin, substantially as denoted by the amino acid sequences as denoted by SEQ ID NO: 25 to 28, respectively, and wherein each of said segments is operably linked to an adjacent segment by a spacer element, wherein each of said spacer elements, which may be identical or different, encodes a signal for proteasomal cleavage selected from the group consisting of: RKSYL, AAY, AVHV, RVTIL and AASRY, substantially as denoted by any one of SEQ ID NO: 20, 23 and 41 to 43, respectively, preferably, said sequence encodes a LTB-breast cancer derived multiepitope fusion protein composed of SEQ ID NOs: 52, 53 and 39, or any functional analogue, variant, equivalent and derivative thereof designated LTB-MEP-Epi.

19. An expression vector encoding a multiepitope fusion protein comprising:
a) a nucleic acid sequences encoding for a multiepitope polypeptide (MEP) which comprises at least two segments, wherein each of said segments, which may be identical or different, encodes a T cell epitope derived from a tumor associated antigen (TAA), and wherein each of said segments is operably linked to an adjacent segment by a spacer element, wherein each of said spacer elements, which may be identical or different, encodes a signal for proteasomal cleavage, operably linked to
b) a nucleic acid sequence encoding LTB (SEQ ID NO: 52), and operably linked to
c) control, promoting and/or regulatory elements;
wherein following proteasomal cleavage, cleavage products of said fusion protein are presented by an APC in the context of MHC Class I and Class II molecules.

20. The expression vector as described in claim 19, wherein the fusion protein encoding sequence is defined in claim 12.

21. An expression system consisting of a host cell transfected with any one of the expression vectors defined in claim 20, wherein said host cell is any one of eukaryotic and prokaryotic cell, preferably a mammalian antigen presenting cell (APC).

22. A fusion protein comprising the LTB peptide sequence fused to a MEP polypeptide, wherein following proteasomal cleavage, cleavage products of said fusion protein are presented by an APC in the context of MHC Class I and Class II molecules and induce B and T cell activation.

23. The fusion protein according to claim 22, wherein said fusion protein comprises a melanoma-associated multiepitope polypeptide fused to LTB, which fusion protein is designated LTB-MEP-Mel and has the amino acid sequence as denoted by SEQ ID NO: 56, or any functional analogue, variant, equivalent and derivative thereof

24. The fusion protein according to claim 22, wherein said fusion protein comprises an epithelial carcinoma derived multiepitope polypeptide fused to LTB, which fusion protein is designated LTB-MEP-Epi composed by the amino acid of sequences SEQ ID NO: 55 and 40, or any functional analogue, variant, equivalent and derivative thereof.

25. A composition for inducing an immune response directed against malignancy in a mammalian subject, comprising as an active ingredient a multiepitope polypeptide (MEP) as defined in claim 1.

26. A composition for inducing an immune response directed against malignancy in a mammalian subject, comprising as an active ingredient at least one of a LTB-MEP fusion protein as defined in claim 22, a recombinant nucleic acid sequence encoding said LTB-MEP fusion protein as defined in claim 12, an expression vector encoding said LTB-MEP fusion protein as defined in claim 19, and an expression system consisting of a host cell transfected with said vectors as defined in claim 21.

27. A pharmaceutical composition for the treatment of a malignant disorder in a mammalian subject, comprising as an active ingredient a multiepitope polypeptide (MEP) as defined in claim 1, and optionally further comprising pharmaceutically acceptable carrier, diluent, excipient, adjuvant and additive.

28. A pharmaceutical composition for the treatment of a malignant disorder in a mammalian subject, comprising as an active ingredient any one of a LTB-MEP fusion protein as defined in claim 22, a recombinant nucleic acid sequence encoding said LTB-MEP fusion protein as defined in claim 12, an expression vector encoding said LTB-MEP fusion protein as defined in claim 19, and a host cell transfected with said vectors as defined in claim 21, optionally further comprising pharmaceutically acceptable carrier, diluent, excipient, adjuvant and additive.

29. The composition according to claim 25, wherein said adjuvant is IgG Fc fragment and/or an enterotoxin, preferably LTB.

30. The composition according to claim 25, wherein said malignancy or malignant disorder is any one of carcinomas, lymphomas, melanomas and sarcomas, preferably, said malignancy or malignant disorder is melanoma.

31. The composition according to claim 30, wherein said mammalian subject is human.

32. The composition according to claim 31, wherein said APC are autologous dendritic cells (DC).

33. A method for conferring immunity against a malignancy in a mammalian subject, comprising the step of administering to said subject a multiepitope polypeptide (MEP) as defined in claim 1, an autologous APC loaded with said MEP or a composition comprising the same, in an amount sufficient to induce in said subject an immune response against said malignancy.

34. A method for conferring immunity against a malignancy in a mammalian subject, comprising the step of administering to said subject a LTB-MEP fusion protein as defined in claim 22, a recombinant nucleic acid sequence encoding said LTB-MEP fusion protein as defined in claim 12, an expression vector encoding said LTB-MEP fusion protein as defined in claim 19, an expression system consisting of a host cell transfected with said vector as defined in claim 21, an autologous APC loaded with said LTB-MEP fusion protein or a composition comprising the same, in an amount sufficient to induce in said subject an immune response against said malignancy.

35. A method for the treatment of a malignant disorder in a mammalian subject in need, comprising the step of administering to said subject a multiepitope polypeptide (MEP) as defined in claim 1, an autologous APC loaded with said MEP or a composition comprising the same, in an amount sufficient to induce in said subject an immune response against said malignancy.

36. A method for the treatment of a malignant disorder in a mammalian subject in need, comprising the step of administering to said subject a LTB-MEP fusion protein as defined in claim 22, a recombinant nucleic acid sequence encoding said LTB-MEP fusion protein as defined in claim 12, an expression vector encoding said LTB-MEP fusion protein as defined in claim 19, an expression system consisting of a host cell transfected with said vector as defined in claim 21, an autologous APC loaded with said LTB-MEP fusion protein or a composition comprising the same, in an amount sufficient to induce in said subject an immune response against said malignancy.

37. The method according to claim 33, wherein said malignancy is selected from the group consisting of carcinomas, lymphomas, melanomas and sarcomas, preferably, said malignancy or a malignant disorder is melanoma.

38. The method according to claim 37, wherein said mammal is human.

39. The method according to claim 38, wherein said host cell transfected with said vector is an autologous APC, preferably, said APC is autologous dendritic cell (DC).

40. A transdermal drug delivery system for the treatment of a malignant disorder by inducing a systemic antigen specific immune response in a mammalian subject in need, comprising as an active ingredient at least the B subunit of the LT protein (LTB) conjugated to or mixed with a multiepitope polypeptide, MEP, as defined in claim 1 which drug delivery system optionally further comprises pharmaceutically acceptable carrier, diluent, excipient, adjuvant and/or additive.

41. The transdermal drug delivery system according to claim 40, wherein said LTB is recombinantly fused to MEP, creating the LTB-MEP fusion protein as defined in claim 22.

42. The transdermal drug delivery system according to claim 40, in the form of an ointment, cream, spray, patches, sustained-release patches or any other suitable transdermal delivery vehicle.

43. The transdermal drug delivery system according to claim 40, wherein said malignancy is selected from the group consisting of carcinomas, lymphomas, melanomas and sarcomas, preferably, said malignancy or a malignant disorder is melanoma.

44. The transdermal drug delivery system according to claim 40, wherein said immune response results in the production of antibodies, helper and cytotoxic T lymphocytes, specific for different antigens associated with said malignancy, comprised in said LTB-MEP fusion protein.

45. A method for the treatment of a malignant disorder in a mammalian subject in need, comprising the step of applying to said subject the transdermal drug delivery system as defined in claim 40, in an amount sufficient to induce in said subject a systemic immune response against said malignancy.

46. The method according to claim 45, wherein said malignancy is selected from the group consisting of carcinomas, lymphomas, melanomas and sarcomas, preferably, said malignancy or a malignant disorder is melanoma.

47. The method according to claim 46, wherein said mammal is human.


SEQUENCE LISTING (57 sequences)

SEQ ID NO: 1 - DNA, 495 bp, Homo sapiens
cccggtaccatgggatcctgccgtaaatcttactacctggaaccgggcccggtgacggtgcgcaaaagctatctgattatggatcaggtgccgtttagcgtgcgtaagtcttacctgtacatggatggcacgatgtctcaggtgcgcaagagctacctggcgggtattggcatcctgaccgtgcgcaagtcttattatctggaaccgggtccggtgaccgttgcgctgctgattatggatcaggtgccgttttctgttgcactgttatatatggatggtacgatgagccaggtggcgttactgctggcgggtatcggtattctgaccgtgagcagcctgtatctggaaccgggtccggtgacggtggcggcgtatattatggatcaggtgccgtttagtgtgtcttctctgtacatggatggcaccatgagtcaggtggcagcgtacctggcgggcatcggcatcctgaccgtttaataaaagcttgaattccgg

SEQ ID NO: 2 - protein, 158 aa, Artificial Sequence
  Recombinant Multiepitope Polypeptide
PGTMGSCRKSYYLEPGPVTVRKSYLIMDQVPFSVRKSYLYMDGTMSQVRKSYLAGIGILTVRKSYYLEPGPVTVALLIMDQVPFSVALLYMDGTMSQVALLLAGIGILTVSSLYLEPGPVTVAAYIMDQVPFSVSSLYMDGTMSQVAAYLAGIGILTV

SEQ ID NO: 3 - DNA, 23 bp, Homo sapiens
cccggtaccatgggatcctgccg

SEQ ID NO: 4 - DNA, 73 bp, Homo sapiens
cataatcagatagcttttgcgcaccgtcaccgggcccggttccaggtagtaagatttacggcaggatcccatg

SEQ ID NO: 5 - DNA, 57 bp, Homo sapiens
agctatctgattatggatcaggtgccgtttagcgtgcgtaagtcttacctgtacatg

SEQ ID NO: 6 - DNA, 50 bp, Homo sapiens
ggtagctcttgcgcacctgagacatcgtgccatccatgtacaggtaagac

SEQ ID NO: 7 - DNA, 49 bp, Homo sapiens
ggtgcgcaagagctacctggcgggtattggcatcctgaccgttcgcaag

SEQ ID NO: 8 - DNA, 64 bp, Homo sapiens
cataatcagcagcgcaacggtcaccggacccggttccagataataagacttgcgaacggtcagg

SEQ ID NO: 9 - DNA, 48 bp, Homo sapiens
gcgctgctgattatggatcaggtgccgttttctgttgcactgttatac

SEQ ID NO: 10 - DNA, 60 bp, Homo sapiens
gatacccgccagcagtaacgccacctggctcatcgtaccatccatgtataacagtgcaac

SEQ ID NO: 11 - DNA, 82 bp, Homo sapiens
gttactgctggcgggtatcggtattctgaccgtgagcagcctgtatctggaaccgggtccggtgacggtggcggcgtatatc

SEQ ID NO: 12 - DNA, 82 bp, Homo sapiens
cgctgccacctgactcatggtgccatccatgtacagagaagacacactaaacggcacctgatccatgatatacgccgccacc

SEQ ID NO: 13 - DNA, 59 bp, Homo sapiens
gagtcaggtggcagcgtacctggcgggcatcggcatcctgaccgtttaataaaagcttg

SEQ ID NO: 14 - DNA, 24 bp, Homo sapiens
ccggaattcaagcttttattaaac

SEQ ID NO: 15 - protein, 9 aa, Homo sapiens
YLEPGPVTV

SEQ ID NO: 16 - protein, 9 aa, Homo sapiens
IMDQVPFSV

SEQ ID NO: 17 - protein, 9 aa, Homo sapiens
YMDGTMSQV

SEQ ID NO: 18 - protein, 9 aa, Homo sapiens
LAGIGILTV

SEQ ID NO: 19 - protein, 4 aa, Homo sapiens
RKSY

SEQ ID NO: 20 - protein, 5 aa, Homo sapiens
RKSYL

SEQ ID NO: 21 - protein, 3 aa, Homo sapiens
ALL

SEQ ID NO: 22 - protein, 3 aa, Homo sapiens
SSL

SEQ ID NO: 23 - protein, 3 aa, Homo sapiens
AAY

SEQ ID NO: 24 - protein, 10 aa, Homo sapiens
KTWGQYWQVL

SEQ ID NO: 25 - protein, 9 aa, Homo sapiens
LLLTVLTVV

SEQ ID NO: 26 - protein, 9 aa, Homo sapiens
NLTISDVSV

SEQ ID NO: 27 - protein, 9 aa, Homo sapiens
NLFETPVEA

SEQ ID NO: 28 - protein, 9 aa, Homo sapiens
GLQHWVPEL

SEQ ID NO: 29 - DNA, 66 bp, Homo sapiens
cgcggatcccgcaagtcttatctgctgctgctgaccgtgctgaccgtggtgcgtaagtcttacctg

SEQ ID NO: 30 - DNA, 54 bp, Homo sapiens
cagataggccgcaaccacggtcagcacggtcagcagcagcaggtaagacttacg

SEQ ID NO: 31 - DNA, 49 bp, Homo sapiens
gttgcggcctatctgctgctgaccgtgctgacggttgttaatctgttcg

SEQ ID NO: 32 - DNA, 46 bp, Homo sapiens
cacatgcacggccgcttccaccggggtttcgaacagattaacaacc

SEQ ID NO: 33 - DNA, 55 bp, Homo sapiens
gcggccgtgcatgtgaacctgtttgaaaccccggtggaagcccgcaaaagctatc

SEQ ID NO: 34 - DNA, 60 bp, Homo sapiens
cagaaaggtcacgcgcgcttccaccggggtttcaaacaggttcagatagcttttgcgggc

SEQ ID NO: 35 - DNA, 81 bp, Homo sapiens
cgcgtgacctttctgggcctgcagcattgggtgccggaactgggcctgcagcattgggtgccggagctgcgcaagagctac

SEQ ID NO: 36 - DNA, 63 bp, Homo sapiens
agagatcgtcagattcagttccggcacccaatgctgcaggcccaggtagctcttgagcagctc

SEQ ID NO: 37 - DNA, 85 bp, Homo sapiens
gaatctgacgatctctgatgtgagcgtgcgtaaatcttatctgaacctgaccattagcgatgtgagcgtggcggcgtctcgctat

SEQ ID NO: 38 - DNA, 60 bp, Homo sapiens
cccaagcttgggttactacacgctcacatcgctaatggtcaggttatagcgagacgccgc

SEQ ID NO: 39 - DNA, 435 bp, Homo sapiens
ctgctgctgaccgtgctgaccgtggtgcgcaaaagctatctgctgctgctgaccgtgctgaccgtggtggcggcgtatctgctgctgaccgtgctgaccgtggtgaacctgtttgaaaccccggtggaagcgcgcaaaagctatctgaacctgtttgaaaccccggtggaagcggcggtgcatgtgaacctgtttgaaaccccggtggaagcgcgcgtgacctttctgggcctgcagcattgggtgccggaactgggcctgcagcattgggtgccggaactgcgcaaaagctatctgggcctgcagcattgggtgccggaactgaacctgaccattagcgatgtgagcgtggcggcgtctcgctataacctgaccattagcgatgtgagcgtgcgcaaaagctatctgaacctgaccattagcgatgtgagcgtg

SEQ ID NO: 40 - protein, 154 aa, Homo sapiens
MRGSRKSYLLLLTVLTVVRKSYLLLLTVLTVVAAYLLLTVLTVVNLFETPVEAAVHVNLFETPVEARKSYLNLFETPVEARVTFLGLQHWVPELGLQHWVPELRKSYLGLQHWVPELNLTISDVSVRKSYLNLTISDVSVAASRYNLTISDVSV

SEQ ID NO: 41 - protein, 4 aa, Homo sapiens
AVHV

SEQ ID NO: 42 - protein, 5 aa, Homo sapiens
RVTIL

SEQ ID NO: 43 - protein, 5 aa, Homo sapiens
AASRY

SEQ ID NO: 44 - DNA, 26 bp, Homo sapiens
cagtatggaaaacgatccccgggtac

SEQ ID NO: 45 - DNA, 22 bp, Homo sapiens
ccggggatcgttttccatactg

SEQ ID NO: 46 - DNA, 32 bp, Homo sapiens
gatccctgcagatctgcggccgctcgaggtac

SEQ ID NO: 47 - DNA, 20 bp, Homo sapiens
gatcctgccgtaaatcttac

SEQ ID NO: 48 - DNA, 16 bp, Homo sapiens
gtaagatttacggcag

SEQ ID NO: 49 - DNA, 32 bp, Homo sapiens; identical to SEQ ID NO: 46

SEQ ID NO: 50 - DNA, 48 bp, Homo sapiens
gatccccgggtacctcgagcggccgcagatctgcagggatcctgccgt

SEQ ID NO: 51 - DNA, 48 bp, Homo sapiens
acggcaggatccctgcagatctgcggccgctcgaggtacccggggatc

SEQ ID NO: 52 - DNA, 369 bp, Escherichia coli
aataaagtaaaatgttatgttttatttacggcgttactatcctctctatgtgcatacggagctccccagtctattacagaactatgttcggaatatcgcaacacacaaatatatacgataaatgacaagatactatcatatacggaatcgatggcaggcaaaagagaaatggttatcattacatttaagagcggcgcaacatttcaggtcgaagtcccgggcagtcaacatatagactcccaaaaaaaagccattgaaaggatgaaggacacattaagaatcacatatctgaccgagaccaaaattgataaattatgtgtatggaataataaaacccccaattcaattgcggcaatcagtatggaaaac

SEQ ID NO: 53 - DNA, 12 bp, Artificial Sequence
  Synthetic Linker
agtatggaaaac

SEQ ID NO: 54 - DNA, 888 bp, Artificial Sequence
  Recombinant Homo sapiens Escherichia coli
aataaagtaaaatgttatgttttatttacggcgttactatcctctctatgtgcatacggagctccccagtctattacagaactatgttcggaatatcgcaacacacaaatatatacgataaatgacaagatactatcatatacggaatcgatggcaggcaaaagagaaatggttatcattacatttaagagcggcgcaacatttcaggtcgaagtcccgggcagtcaacatatagactcccaaaaaaaagccattgaaaggatgaaggacacattaagaatcacatatctgaccgagaccaaaattgataaattatgtgtatggaataataaaacccccaattcaattgcggcaatcagtatggaaaacgatccccgggtacctcgagcggccgcagatctgcagggatcctgccgtaaatcttactacctggaaccgggcccggtgacggtgcgcaaaagctatctgattatggatcaggtgccgtttagcgtgcgtaagtcttacctgtacatggatggcacgatgtctcaggtgcgcaagagctacctggcgggtattggcatcctgaccgtgcgcaagtcttattatctggaaccgggtccggtgaccgttgcgctgctgattatggatcaggtgccgttttctgttgcactgttatatatggatggtacgatgagccaggtggcgttactgctggcgggtatcggtattctgaccgtgagcagcctgtatctggaaccgggtccggtgacggtggcggcgtatattatggatcaggtgccgtttagtgtgtcttctctgtacatggatggcaccatgagtcaggtggcagcgtacctggcgggcatcggcatcctgaccgtttaataaaagcttgaattccgg

SEQ ID NO: 55 - protein, 123 aa, Escherichia coli
NKVKCYVLFTALLSSLCAYGAPQSITELCSEYRNTQIYTINDKILSYTESMAGKREMVIITFKSGATFQVEVPGSQHIDSQKKAIERMKDTLRITYLTETKIDKLCVWNNKTPNSIAAISMEN

SEQ ID NO: 56 - protein, 289 aa, Artificial Sequence
  Recombinant Homo sapiens Escherichia coli
NKVKCYVLFTALLSSLCAYGAPQSITELCSEYRNTQIYTINDKILSYTESMAGKREMVIITFKSGATFQVEVPGSQHIDSQKKAIERMKDTLRITYLTETKIDKLCVWNNKTPNSIAAISMENDPRVPRAAADLQGSCRKSYYLEPGPVTVRKSYLIMDQVPFSVRKSYLYMDGTMSQVRKSYLAGIGILTVRKSYYLEPGPVTVALLIMDQVPFSVALLYMDGTMSQVALLLAGIGILTVSSLYLEPGPVTVAAYIMDQVPFSVSSLYMDGTMSQVAAYLAGIGILTV

SEQ ID NO: 57 - protein, 155 aa, Artificial Sequence
  Recombinant Multiepitope Polypeptide
MGSCRKSYYLEPGPVTVRKSYLIMDQVPFSVRKSYLYMDGTMSQVRKSYLAGIGILTVRKSYYLEPGPVTVALLIMDQVPFSVALLYMDGTMSQVALLLAGIGILTVSSLYLEPGPVTVAAYIMDQVPFSVSSLYMDGTMSQVAAYLAGIGILTV